(19) United States
(12) Patent Application Publication
(10) Pub. No.: US 2022/0390349 A1
(43) Pub. Date: Dec. 8, 2022

(54) METHODS AND SYSTEMS FOR CLASSIFYING FLOW CYOMETER DATA

(71) Applicant: Becton, Dickinson and Company, Franklin Lakes, NJ (US)

(72) Inventors: David Alexander Roberts, San Jose, CA (US); Christopher J. Wolf, San Jose, CA (US); Keegan Owsley, Campbell, CA (US)

(21) Appl. No.: 17/699,865

(22) Filed: Mar. 21, 2022

Related U.S. Application Data
(60) Provisional application No. 63/196,848, filed on Jun. 4, 2021.

(51) Int. Cl. G01N 15/14; G01N 33/49; G01N 33/493

(52) U.S. Cl. CPC G01N 15/1429; G01N 33/4915; G01N 33/493; G01N 2015/149; G01N 2015/1481

(57) ABSTRACT

Methods of classifying flow cytometer data are provided. Methods of interest include receiving a first gate and flow cytometer data, expanding the first gate to generate a second gate, and determining sets of flow cytometer data encompassed by each of the first gate and the second gate to classify the flow cytometer data. In embodiments, methods also involve recording a subset of the classified flow cytometer data and optionally adjusting the first and/or second gates based on the recorded data. In some cases, the subject methods include sorting particles associated with the classified flow cytometer data based on the first and second gates. Systems and computer-readable storage media for practicing the invention are also provided.

### CROSS-REFERENCE TO RELATED APPLICATIONS

Pursuant to 35 U.S.C. § 119 (e), this application claims priority to the filing date of U.S. Provisional Patent Application Ser. No. 63/196,848 filed Jun. 4, 2021; the disclosure of which application is incorporated herein by reference in their entirety.

### INTRODUCTION

The characterization of analytes in biological fluids has become an important part of biological research, medical diagnoses and assessments of overall health and wellness of a patient. Detecting analytes in biological fluids, such as human blood or blood derived products, can provide results that may play a role in determining a treatment protocol of a patient having a variety of disease conditions.

Flow cytometry is a technique used to characterize and often times sort biological material, such as cells of a blood sample or particles of interest in another type of biological or chemical sample. A flow cytometer typically includes a sample reservoir for receiving a fluid sample, such as a blood sample, and a sheath reservoir containing a sheath fluid. The flow cytometer transports the particles (including cells) in the fluid sample as a cell stream to a flow cell, while also directing the sheath fluid to the flow cell. To characterize the components of the flow stream, the flow stream is irradiated with light. Variations in the materials in the flow stream, such as morphologies or the presence of fluorescent labels, may cause variations in the observed light and these variations allow for characterization and separation. To characterize the components in the flow stream, light must impinge on the flow stream and be collected. Light sources in flow cytometers can vary and may include one or more broad spectrum lamps, light emitting diodes as well as single wavelength lasers. The light source is aligned with the flow stream and an optical response from the illuminated particles is collected and quantified.

Isolation of biological particles has been achieved by adding a sorting or collection capability to flow cytometers. Particles in a segregated stream, detected as having one or more desired characteristics, are individually isolated from the sample stream by mechanical or electrical removal. A common flow sorting technique utilizes drop sorting in which a fluid stream containing linearly segregated particles is broken into drops. The drops containing particles of interest are electrically charged and deflected into a collection tube by passage through an electric field. Typically, the linearly segregated particles in the stream are characterized as they pass through an observation point situated just below the nozzle tip. Once a particle is identified as meeting one or more desired criteria, the time at which it will reach the drop break-off point and break from the stream in a drop can be predicted. Ideally, a brief charge is applied to the fluid stream just before the drop containing the selected particle breaks from the stream and then grounded immediately after the drop breaks off. The drop to be sorted maintains an electrical charge as it breaks off from the fluid stream, and all other drops are left un-charged.

The parameters measured using a flow cytometer typically include light at the excitation wavelength scattered by the particle in a narrow angle along a mostly forward direction, referred to as forward-scatter (FSC), the excitation light that is scattered by the particle in an orthogonal direction to the excitation laser, referred to as side-scatter (SSC), and the light emitted from fluorescent molecules in one or more detectors that measure signal over a range of spectral wavelengths, or by the fluorescent dye that is primarily detected in that specific detector or array of detectors. Different cell types can be identified by their light scatter characteristics and fluorescence emissions resulting from labeling various cell proteins or other constituents with fluorescent dye-labeled antibodies or other fluorescent probes.

Flow cytometers may further comprise means for recording the measured data and analyzing the data. For example, data storage and analysis may be carried out using a computer connected to the detection electronics. For example, the data can be stored in tabular form, where each row corresponds to data for one particle, and the columns correspond to each of the measured features. The use of standard file formats, such as an “FCS” file format, for storing data from a particle analyzer facilitates analyzing data using separate programs and/or machines. Using current analysis methods, the data typically are displayed in 1-dimensional histograms or 2-dimensional (2D) plots for ease of visualization, but other methods may be used to visualize multidimensional data.

While flow cytometer data generally contains numerous data points (i.e., events), it is often the case that only a certain portion of the flow cytometer data is of interest to the user. For example, a sample may include monocytes, granulocytes and lymphocytes, but a user may only be interested in lymphocyte data. In conventional approaches to classifying flow cytometer data, a user must often determine an ideal boundary (i.e., gate) for separating events of interest from other events. Because an ideal boundary is often not immediately apparent, users may be required to experiment with increasing or decreasing the size of the storage gate in order to achieve a desired level of purity.

### SUMMARY

Because conventional approaches for gating flow cytometer data involve considerable ambiguity regarding an ideal boundary between different populations of data, the inventors have realized that new methods and systems for classifying flow cytometer data are needed. Embodiments of the invention satisfy this and other needs.

Aspects of the invention include methods for classifying flow cytometer data. Methods of interest include receiving a first gate having a set of vertices and flow cytometer data, expanding the first gate to generate a second gate, and determining sets of flow cytometer data encompassed by each of the first and second gates to classify the flow cytometer data. In some cases, expanding the first gate includes adjusting each vertex of the first gate such that the horizontal and vertical differences of the vertices from the first gate's centroid is increased by a percentage. In other cases, expanding the first gate to generate the second gate includes Minkowski addition (e.g., summing the vertices of the first gate with the vertices of an expansion circle). Embodiments of the subject methods also include recording a subset of the classified flow cytometer data. In certain embodiments, the recorded subset of flow cytometer data includes the set of flow cytometer data that is encompassed by the second gate. In other embodiments, the recorded subset of classified flow cytometer data includes a random sample of the flow cytometer data within a set difference of the set of flow cytometer data encompassed by the second gate and the set of flow cytometer data encompassed by the first gate. The random sample may, in some cases, include a random sample of the flow cytometer data within a given distance from the first gate. In certain instances, the recorded subset of classified flow cytometer data includes the set union of the random sample and the set of flow cytometer data encompassed by the first gate. Embodiments of the method further include processing the recorded subset of classified flow cytometer data with a dimensionality reduction algorithm (e.g., tSNE) and associating the recorded subset of classified flow cytometer data with a phenotype. In some cases, methods additionally include adjusting the vertices of the first and/or second gates based on an analysis of the recorded subset of flow cytometer data. In some embodiments, methods also include sorting particles via a sorting flow cytometer based on the first and second gates. Computer-readable storage media for practicing the subject methods are also provided.

Aspects of the disclosure further include systems for practicing the subject invention. Systems of interest include a particle analyzer (e.g., flow cytometer) for obtaining flow cytometer data and a processor configured to: receive a first gate and flow cytometer data, expand the first gate to generate a second gate, and determine sets of flow cytometer data encompassed by each of the first gate and the second gate to classify the flow cytometer data. In some cases, expanding the first gate includes adjusting each vertex of the first gate such that the horizontal and vertical differences of the vertices from the first gate's centroid is increased by a percentage. In other cases, expanding the first gate to generate the second gate includes Minkowski addition (e.g., summing the vertices of the first gate with the vertices of an expansion circle). In certain embodiments, the processor also includes instructions for recording a subset of classified flow cytometer data. In certain embodiments, the recorded subset of flow cytometer data includes the set of flow cytometer data that is encompassed by the second gate. In other embodiments, the recorded subset of classified flow cytometer data includes a random sample of the flow cytometer data within a set difference of the set of flow cytometer data encompassed by the second gate and the set of flow cytometer data encompassed by the first gate. The random sample may, in some cases, include a random sample of the flow cytometer data within a given distance from the first gate. In certain instances, the recorded subset of classified flow cytometer data includes the set union of the random sample and the set of flow cytometer data encompassed by the first gate. In some instances, the flow cytometer in the present systems is a sorting flow cytometer that is configured to differentially sort particles in a sample. In such instances, the sorting flow cytometer may be configured to sort particles based on the first and second gates. For example, the sorting flow cytometer may be configured to sort particles associated with the set of flow cytometer data encompassed by the first gate into a first collection vessel, and sort particles associated with flow cytometer data encompassed by the set difference of the set of flow cytometer data encompassed by the second gate and the set of flow cytometer data encompassed by the first gate into a second collection vessel.

### BRIEF DESCRIPTION OF THE FIGURES

The invention may be best understood from the following detailed description when read in conjunction with the accompanying drawings. Included in the drawings are the following figures:

FIG. 1 depicts the expansion of a first gate to generate a second gate according to certain embodiments.

FIG. 2 depicts the expansion of a first gate via Minkowski addition according to certain embodiments.

FIG. 3 illustrates the determination of a subset of flow cytometer data to be recorded according to certain embodiments.

FIG. 4 depicts a flowchart for classifying flow cytometer data according to certain embodiments.

FIG. 5 depicts a functional block diagram of a flow cytometric system according to certain embodiments.

FIG. 6 depicts a sorting control system according to certain embodiments.

FIG. 7A-B depict a schematic drawing of a particle sorter system according to certain embodiments.

FIG. 8 depicts a block diagram of a computing system according to certain embodiments.

### DETAILED DESCRIPTION

Methods of classifying flow cytometer data are provided. Methods of interest include receiving a first gate and flow cytometer data, expanding the first gate to generate a second gate, and determining sets of flow cytometer data encompassed by each of the first gate and the second gate to classify the flow cytometer data. In embodiments, methods also involve recording a subset of the classified flow cytometer data and optionally adjusting the first and/or second gates based on the recorded data. In some cases, the subject methods include sorting particles associated with the classified flow cytometer data based on the first and second gates. Systems and computer-readable storage media for practicing the invention are also provided.

Before the present invention is described in greater detail, it is to be understood that this invention is not limited to particular embodiments described, as such may, of course, vary. It is also to be understood that the terminology used herein is for the purpose of describing particular embodiments only, and is not intended to be limiting, since the scope of the present invention will be limited only by the appended claims.

Where a range of values is provided, it is understood that each intervening value, to the tenth of the unit of the lower limit unless the context clearly dictates otherwise, between the upper and lower limit of that range and any other stated or intervening value in that stated range, is encompassed within the invention. The upper and lower limits of these smaller ranges may independently be included in the smaller ranges and are also encompassed within the invention, subject to any specifically excluded limit in the stated range. Where the stated range includes one or both of the limits, ranges excluding either or both of those included limits are also included in the invention.

Certain ranges are presented herein with numerical values being preceded by the term “about.” The term “about” is used herein to provide literal support for the exact number that it precedes, as well as a number that is near to or approximately the number that the term precedes. In determining whether a number is near to or approximately a specifically recited number, the near or approximating unrecited number may be a number which, in the context in which it is presented, provides the substantial equivalent of the specifically recited number.

Unless defined otherwise, all technical and scientific terms used herein have the same meaning as commonly understood by one of ordinary skill in the art to which this invention belongs. Although any methods and materials similar or equivalent to those described herein can also be used in the practice or testing of the present invention, representative illustrative methods and materials are now described.

All publications and patents cited in this specification are herein incorporated by reference as if each individual publication or patent were specifically and individually indicated to be incorporated by reference and are incorporated herein by reference to disclose and describe the methods and/or materials in connection with which the publications are cited. The citation of any publication is for its disclosure prior to the filing date and should not be construed as an admission that the present invention is not entitled to antedate such publication by virtue of prior invention. Further, the dates of publication provided may be different from the actual publication dates which may need to be independently confirmed.

It is noted that, as used herein and in the appended claims, the singular forms “a”, “an”, and “the” include plural referents unless the context clearly dictates otherwise. It is further noted that the claims may be drafted to exclude any optional element. As such, this statement is intended to serve as antecedent basis for use of such exclusive terminology as “solely,” “only” and the like in connection with the recitation of claim elements, or use of a “negative” limitation.

As will be apparent to those of skill in the art upon reading this disclosure, each of the individual embodiments described and illustrated herein has discrete components and features which may be readily separated from or combined with the features of any of the other several embodiments without departing from the scope or spirit of the present invention. Any recited method can be carried out in the order of events recited or in any other order which is logically possible.

While the system and method has or will be described for the sake of grammatical fluidity with functional explanations, it is to be expressly understood that the claims, unless expressly formulated under 35 U.S.C. § 112, are not to be construed as necessarily limited in any way by the construction of “means” or “steps” limitations, but are to be accorded the full scope of the meaning and equivalents of the definition provided by the claims under the judicial doctrine of equivalents, and in the case where the claims are expressly formulated under 35 U.S.C. § 112 are to be accorded full statutory equivalents under 35 U.S.C. § 112.

### Methods of Classifying Flow Cytometer Data

As discussed above, aspects of the invention include methods for classifying flow cytometer data. By “flow cytometer data” it is meant information regarding the characteristics of sample particles that has been collected by any number of detectors in a particle analyzer. As discussed herein, a “particle analyzer” is an analytical tool (e.g., flow cytometer) that enables the characterization of particles on the basis of certain (e.g., optical) parameters. By “particle”, it is meant a discrete component of a biological sample such as a molecule, analyte-bound bead, individual cell, or the like.

Methods of interest include classifying one or more population clusters based on determined parameters (e.g., fluorescence) of analytes (e.g., particles) in a sample. As used herein, a “population”, or “subpopulation” of analytes, such as cells or other particles, generally refers to a group of analytes that possess properties (for example, optical, impedance, or temporal properties) with respect to one or more measured parameters such that measured parameter data form a cluster in the data-space. The data obtained from an analysis of cells (or other particles) by flow cytometry are often multidimensional, where each cell corresponds to a point in a multidimensional space defined by the parameters measured. In embodiments, data is comprised of signals from a plurality of different parameters, such as, for instance 2 or more, 3 or more, 4 or more, 5 or more, 6 or more, 7 or more, 8 or more, 9 or more, 10 or more, and including or more. Thus, populations are recognized as clusters in the data. Conversely, each data cluster generally is interpreted as corresponding to a population of a particular type of cell or analyte, although clusters that correspond to noise or background typically also are observed. A cluster may be defined in a subset of the dimensions, e.g., with respect to a subset of the measured parameters (e.g., fluorochromes), which corresponds to populations that differ in only a subset of the measured parameters or features extracted from the measurements of the sample.

Aspects of the subject methods include receiving a first gate having a set of vertices. As discussed herein, a “gate” generally refers to a classifier boundary identifying a subset of data of interest. In cytometry, a gate can bound a group (i.e., population) of events of particular interest. In other words, a gate defines a boundary for classifying populations of flow cytometer data. In embodiments, a gate identifies flow cytometer data exhibiting the same or similar set of parameters. In addition, “gating” generally refers to the process of classifying the data using a defined gate for a given set of data, where the gate can be one or more regions of interest combined with Boolean logic. Examples of methods for gating have been described in, for example, U.S. Pat. Nos. 4,845,653; 5,627,040; 5,739,000; 5,795,727; 5,962,238; 6,014,904; 6,944,338; and 8,990,047; each of which is incorporated herein by reference.

In some embodiments, the first gate is a gate that has been drawn by a user. In such embodiments, a user may define the boundaries of a region (e.g., in two-dimensional space) within which flow cytometer data may be assigned a particular classification. For example, drawing a first gate may include superimposing a polygon onto a two-dimensional plot representing flow cytometer data. In other embodiments, the first gate is pre-established by others of skill in cytometry. For example, the first gate may be received from a database of gates that have been employed in previous attempts to classify flow cytometer data. In some embodiments, the first gate is defined by a set of vertices. As discussed herein, “vertices” are points within multidimensional (e.g., two-dimensional) space that, when connected, form the boundaries of a gate.

In embodiments, methods include receiving flow cytometer data, calculating parameters of each analyte, and clustering together analytes based on the calculated parameters. For example, an experiment may include particles labeled by several fluorophores or fluorescently labeled antibodies, and groups of particles may be defined by populations corresponding to one or more fluorescent measurements. In the example, a first group may be defined by a certain range of light scattering for a first fluorophore, and a second group may be defined by a certain range of light scattering for a second fluorophore. If the first and second fluorophores are represented on an x and y axis, respectively, two different color-coded populations might appear to define each group of particles, if the information was to be graphically displayed. How the data is to be represented is determined by the location of each data point (i.e., event) relative to the set of vertices defining each gate. Any number of analytes may be classified within a gate, including 5 or more analytes, such as 10 or more analytes, such as 50 or more analytes, such as 100 or more analytes, such as 500 analytes and including 1000 or more analytes. In certain embodiments, the method groups together in a cluster rare events (e.g., rare cells in a sample, such as cancer cells) detected in the sample. In these embodiments, the analyte clusters generated may include 10 or fewer analytes, such as 9 or fewer and including 5 or fewer assigned analytes.

Flow cytometer data may be received from any suitable source. In some embodiments, flow cytometer data is received from the memory of a storage device. In such embodiments, flow cytometer data may have been previously generated and saved in the memory of the storage device for subsequent recall and analysis. In other embodiments, the flow cytometer data is received in real time. Put another way, flow cytometer data generated during the operation of a flow cytometer may subsequently (e.g., immediately) populate the data-space (e.g., two-dimensional plot) having the first gate. In some cases, the flow cytometer may be operated to generate data until a recording criterion is satisfied. The “recording criterion” discussed herein is a condition that, when met, precipitates the termination of flow cytometer operation and data collection. Any suitable recording criterion may be employed. In certain cases, the recording criterion is a time limit. Where the recording criterion is a time limit, flow cytometer data collection ceases after a prescribed amount of time (e.g., ranging from seconds to 3 hours) has elapsed. In additional cases, the recording criterion is a total number of events. In such instances, flow cytometer data collection ceases after a certain number of particles (e.g., prescribed by the user) have been analyzed. In still additional instances, the recording criterion is a number of events within a population. Flow cytometer data collection may, in such instances, cease after a certain number of particles (e.g., prescribed by the user) within a particular population (e.g., exhibiting a certain phenotype) have been analyzed.

In certain embodiments, the particles are detected and uniquely identified by exposing the particles to excitation light and measuring the fluorescence of each particle in one or more detection channels, as desired. Fluorescence emitted in detection channels used to identify the particles and binding complexes associated therewith may be measured following excitation with a single light source, or may be measured separately following excitation with distinct light sources. If separate excitation light sources are used to excite the particle labels, the labels may be selected such that all the labels are excitable by each of the excitation light sources used.

In embodiments, the flow cytometer data is received from a forward-scattered light detector. Forward-scattered light detectors of interest yield information regarding the overall size of a particle. In embodiments, the flow cytometer data is received from a side-scattered light detector. Side-scattered light detectors of interest detect refracted and reflected light from the surfaces and internal structures of the particle, which tends to increase with increasing particle complexity of structure. In embodiments, the flow cytometer data is received from a fluorescent light detector. Fluorescent light detectors of interest are configured to detect fluorescence emissions from fluorescent molecules, e.g., labeled specific binding members (such as labeled antibodies that specifically bind to markers of interest) associated with the particle in the flow cell. In certain embodiments, methods include detecting fluorescence from the sample with one or more fluorescence detectors, such as 2 or more, such as 3 or more, such as 4 or more, such as 5 or more, such as 6 or more, such as 7 or more, such as 8 or more, such as 9 or more, such as 10 or more, such as 15 or more and including 25 or more fluorescence detectors.

Methods in certain embodiments also include data acquisition, analysis and recording, such as with a computer, wherein multiple data channels record data from each detector for the light scatter and fluorescence emitted by each particle as it passes through the sample interrogation region of the particle sorting module. In these embodiments, analysis includes classifying and counting particles such that each particle is present as a set of digitized parameter values. The subject systems may be set to trigger on a selected parameter in order to distinguish the particles of interest from background and noise. “Trigger” refers to a preset threshold for detection of a parameter and may be used as a means for detecting passage of a particle through the light source. Detection of an event that exceeds the threshold for the selected parameter triggers acquisition of light scatter and fluorescence data for the particle. Data is not acquired for particles or other components in the medium being assayed which cause a response below the threshold. The trigger parameter may be the detection of forward-scattered light caused by passage of a particle through the light beam. The flow cytometer then detects and collects the light scatter and fluorescence data for the particle. The data recorded for each particle is analyzed in real time or stored in a data storage and analysis means, such as a computer, as desired.

After the first gate and flow cytometer data have been received, methods further include expanding the first gate to generate a second gate. By “expanding” the first gate, it is meant that the region of data-space encompassed by the gate is increased. Any convenient mechanism may be employed for gate expansion. In some embodiments, the first gate is expanded based on the distance of one or more gate vertices, and in some instances each gate vertex, to the centroid of the gate. The term “centroid” is employed in its conventional sense to refer to the geometric center of the gate. In some embodiments, methods of interest include calculating the centroid of the first gate. The centroid may be calculated by, for example, determining the arithmetic mean position of every vertex of the first gate. Following the calculation of the centroid, embodiments of the method further include adjusting one or more gate vertices, and in some instances each vertex of the first gate, such that the horizontal and vertical differences of the vertices from the centroid is increased by a percentage. In other words, the distance separating each vertex from the centroid is increased. Any suitable percentage may be employed for expanding the first gate. For example, the percentage may range from 0.5% to 25%, such as 1% to 20%, and including 2% to 10%.

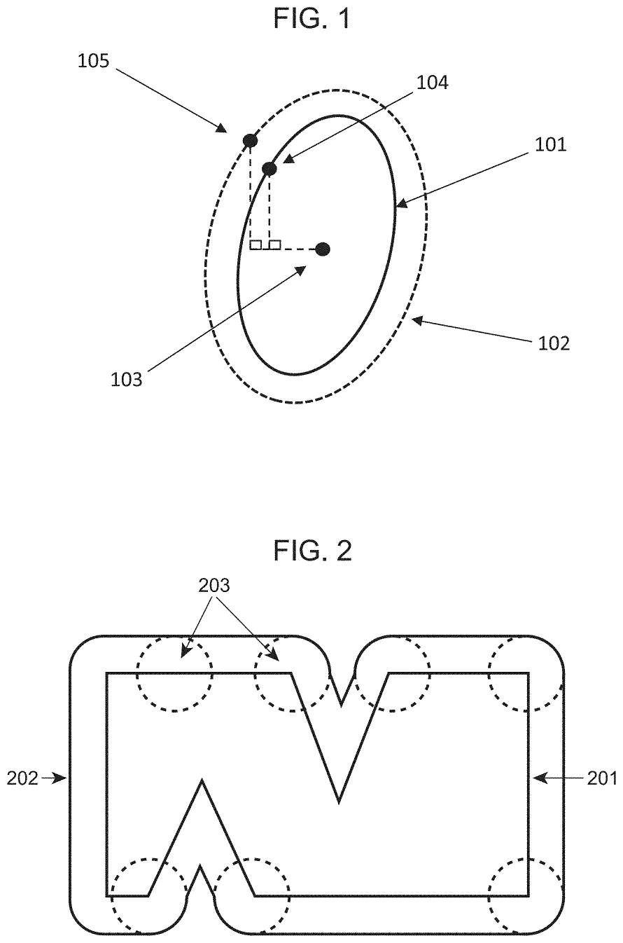

FIG. 1 depicts the expansion of the first gate by increasing the distance of gate vertices from the centroid of the gate. As shown in FIG. 1, first gate 101 is expanded so that second gate 102 is generated. In the process of expanding first gate 101, the distance separating vertex 104 on first gate 101 from the centroid 103 is increased such that an expanded vertex 105 is produced. In order to produce expanded vertex 105, the horizontal and vertical differences (shown as dashed lines) of the vertex 104 from the centroid 103 is increased by a percentage. Although not depicted in FIG. 1, every vertex in first gate 101 is expanded in an identical manner such that second gate 102 is generated.

In additional embodiments, expanding the first gate to generate a second gate includes Minkowski addition. In such embodiments, the set of vertices constituting the first gate is summed with the vertices of an expansion object in order to generate the second gate. By “expansion object” it is meant a geometric figure having characteristics (i.e., size and shape) that are desirable for imparting to the first gate to generate the second gate. In order to expand the first gate by a given magnitude, an expansion object having a particular size may be selected. In other words, the extent to which the first gate is expanded is proportional to the size of the expansion object. Any convenient geometric figure may be employed as the expansion object, including, but not limited to, a circle, ellipse, crescent, triangle, square or rectangle, as well as irregular shapes, e.g., a parabolic bottom portion coupled to a planar top portion. In some embodiments, the expansion object is an expansion circle. For example, where A represents a set of vertices (i.e., position vectors) constituting the first gate and B represents a set of vertices (i.e., position vectors) constituting the expansion object (e.g., expansion circle), the vertices of the second gate may be calculated by adding each vertex in A to each vertex in B. Put another way, the vertices of the second gate may be found in the set resulting from A+B={a+b|a∈A, b∈B}. The set of vertices may subsequently be rendered on a two-dimensional plot and connected such that the second gate is produced and visually represented.

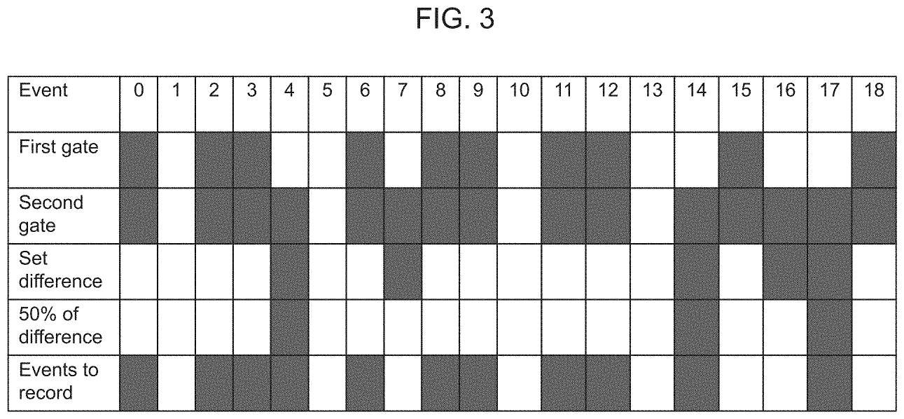

FIG. 2 depicts the expansion of a first gate via Minkowski addition. As shown in FIG. 2, first gate 201 is expanded such that second gate 202 is generated. Each vertex in first gate 201 is summed with the vertices of an expansion circle 203 (e.g., as discussed above). The radius of the expansion circles 203 determines the extent to which first gate 201 is expanded. Due to the curvilinear nature of expansion circles 203, second gate 202 possesses rounded contours in some regions where first gate possesses more sharp edges

Following the generation of the second gate, aspects of the invention further include determining sets of flow cytometer data encompassed by each of the first gate and the second gate to classify the flow cytometer data. In other words, the flow cytometer data that is enclosed by the boundaries of the first gate constitute a first set of flow cytometer data, while the flow cytometer data that is enclosed by the boundaries of the second gate constitute a second set of flow cytometer data. Because the second gate is an expanded version of the first gate and consequently also encompasses every data point within said first gate, the second set of flow cytometer includes the first set of flow cytometer data in addition to data points that lie between the boundaries of the first gate and the second gate. Accordingly, flow cytometer data in some embodiments may be described as “classified” when its relationship with respect to either or both of the first gate and the second gate is established.

In some embodiments, classifying the flow cytometer data includes determining a phenotype associated with the particles (e.g., cells) being irradiated in a particle analyzer. In some cases, a phenotype is determined for a population or subpopulation of flow cytometer data points encompassed by the first and/or second gates. In other words, all of the flow cytometer data encompassed by the first and/or second gates may be associated with a particular subtype or phenotype of particle. Phenotypes may be determined based on the positivity or negativity of the flow cytometer data in the relevant population or subpopulation with respect to any number of different parameters. For example, where the analyzed particles include one or more fluorochromes, the phenotype of a population of flow cytometer data may be determined by assessing the positivity or negativity of the group of particles with respect to each fluorochrome. In certain embodiments, populations of flow cytometer data are their status relative to a hierarchy. A “hierarchy” as described herein defines the criteria by which flow cytometer data is grouped into a particular population and associated with a phenotype. In some embodiments, the hierarchy establishes the shared characteristics of data points that are positive or negative for the same parameters. For example, a hierarchy for clustering T cells might proceed by determining the positivity or negativity of the cells with respect to the presence of CD4 and CD8. A cell that is positive for CD4 but negative for CD8 is a “CD4 T Cell”, while a cell that is positive for both markers is a “Double Positive T Cell”, and so forth.

Embodiments of the invention further include recording a subset of the classified flow cytometer data. By “recording a subset” it is meant selecting a portion of the classified flow cytometer data for storage and/or further analysis. The recorded subset of flow cytometer data may be employed to, for example, assess the suitability of the first gate, the second gate or both for accurately gating flow cytometer data belonging to a certain population. A user may subsequently examine the recorded subset of flow cytometer data and adjust the gate boundaries accordingly.

Any suitable subset of the classified flow cytometer data may be recorded. In certain cases, the recorded subset of classified flow cytometer data includes the set of flow cytometer data encompassed by the second gate. In other words, all of the flow cytometer data within the boundaries of the second gate is recorded, including the flow cytometer data encompassed by the first gate. Where it is desirable to generate a recorded subset of flow cytometer data that includes the set of flow cytometer data encompassed by the second gate, certain embodiments of the method include associating all of the data in the recorded subset with a relevant phenotype, i.e., each data point is determined to be a part of a given subtype of particle.

In additional embodiments, the recorded subset of classified flow cytometer data includes a random sample of the flow cytometer data within a set difference of the set of flow cytometer data encompassed by the second gate and the set of flow cytometer data encompassed by the first gate. The term “set difference” described herein refers to the set of flow cytometer data that is encompassed by the second gate but is not encompassed by the first gate. For example, where A represents the set of flow cytometer data encompassed by the first gate and B represents the set of flow cytometer data encompassed by the second gate, the set difference is determined by B\A={x∈B|x∉A}. Following the generation of the set difference, a random sample (e.g., a simple random sample) may be obtained from the set difference. The random sample may be of any desirable size. In some embodiments, the random sample includes a percentage of the classified flow cytometer data within the set difference. In other words, the number of data points within the random sample may be equal to a percentage of the number of data points within the set difference. Any convenient percentage may be employed. In certain cases, the percentage of the classified flow cytometer data within the set difference ranges from 1% to 99%, such as 30% to 70% and including 40% to 60%. In some embodiments, the percentage is 50%. In other embodiments, the random sample includes a proportion of the classified flow cytometer data within the set difference that is determined relative to the number of datapoints within the set of flow cytometer data encompassed by the first gate. In such embodiments, the number of data points within the random sample may be equal to a percentage of the number of data points that are encompassed by the first gate. For example, in some cases, the percentage the number of data points encompassed by the first gate may range from 1% to 99%, such as 30% to 70% and including 40% to 60%.

In further instances, the recorded subset of classified flow cytometer data includes a random sample of the flow cytometer data within a given distance from the first gate—either in addition to or instead of the recorded subset of flow cytometer data determined as described above. Any convenient distance from the first gate may be employed. In some instances, the distance from the first gate is determined by the user, for example, by drawing a region around the first gate or entering a value for the distance. In certain cases, the sample of flow cytometer data is determined by stratified sampling. As discussed herein, “stratified sampling” refers to a statistical technique involving sampling from a partitioned population. In such embodiments, flow cytometer data existing between within a particular distance from the first gate may be considered a first stratum. The flow cytometer data existing between the first distance and a second, farther, distance may be considered a second stratum, and so on. A random sample may be obtained from each stratum to generate a recorded subset of flow cytometer data. In some instances, the first stratum may be weighted more heavily in the sampling relative to second and subsequent strata due to the proximity of the first stratum to the population of interest as defined by the first gate.

In still further instances, the recorded subset of classified flow cytometer data includes a random sample of the universal set of flow cytometer data in addition to the recorded subset of flow cytometer data determined as described above. By “universal set” of flow cytometer data, it is meant the entirety of the received flow cytometer data.

In some embodiments, the recorded subset of classified flow cytometer data includes the set union of the random sample and the set of flow cytometer data encompassed by the first gate. The term “set union” is employed in its conventional sense to describe a set that includes every element of a collection of sets. The random sample used in the computation of the set union may be obtained by any of the mechanisms described above (e.g., a percentage of the classified flow cytometer data within the set difference, a random sample of the flow cytometer data within a given distance from the first gate, etc.). In certain embodiments, the random sample used in the computation of the set union is a random sample of the flow cytometer data within a set difference of the set of flow cytometer data encompassed by the second gate and the set of flow cytometer data encompassed by the first gate. For example, where A represents the set of flow cytometer data encompassed by the first gate and B represents the random sample, the set union is determined by A∪B={x: x∈A or x∈B}.

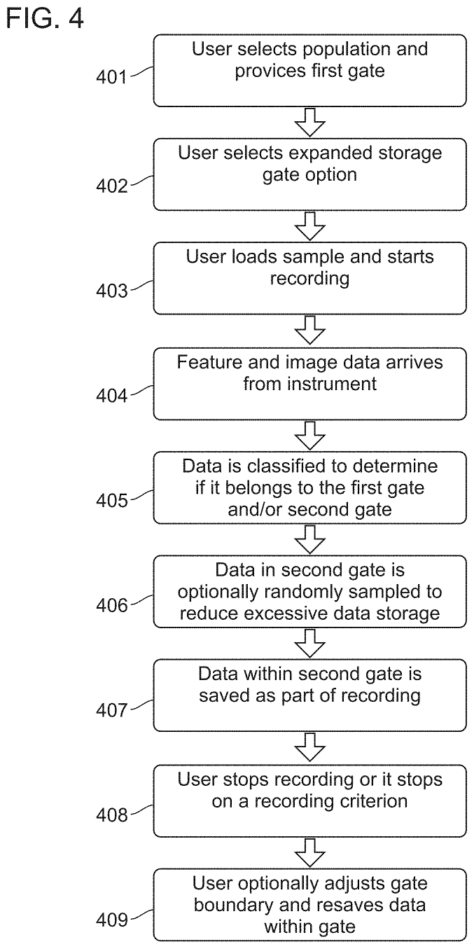

FIG. 3 presents a table depicting the determination of a recorded subset of flow cytometer data. In the “first gate” row, solid blocks indicate the presence of flow cytometer data (i.e. events) that are classified as being encompassed by the first gate. In the “second gate” row, solid blocks indicate the presence of flow cytometer data that are classified as being encompassed by the second (i.e., expanded) gate. Because the second gate is an expanded version of the first gate, the second gate encompasses the flow cytometer data within the first gate as well as additional events. In the “set difference” row, solid blocks indicate the presence of flow cytometer data encompassed by the second gate but not the first gate. In the “50% of difference” row, solid blocks indicate that a random sample was obtained from 50% of the flow cytometer data within the set difference. In the “events to record row”, solid blocks indicate that events encompassed by the first gate are combined (i.e., in a set union) with the random sample obtained from 50% of the flow cytometer data within the set difference in order to generate the recorded subset of flow cytometer data.

After a subset of classified flow cytometer data has been obtained for recordation, embodiments of the method include processing the recorded subset of classified flow cytometer data with a dimensionality reduction algorithm. The term “dimensionality reduction” is used herein in its conventional sense to refer to manipulating a dataset such that the number of different variables under consideration are reduced. Any suitable technique for dimensionality reduction may be employed. In some embodiments, dimensionality reduction includes performing a principal component analysis (PCA) that maps higher dimensional data onto lower dimensional space (e.g., two dimensions) such that the variance of the data in the lower dimensional space is maximized. Any suitable algorithm for dimensionality reduction may be used. In some embodiments, dimensionality reduction is performed by a t-Distributed Stochastic Neighbor Embedding (t-SNE) algorithm. The t-SNE algorithm is described in Laurens van der Maaten & Geoffrey Hinton. Visualizing Data using t-SNE. Journal of Machine Learning Research, 2008; herein incorporated by reference. In some embodiments, dimensionality reduction is performed by a Uniform Manifold Approximation and Projection (UMAP) algorithm. The UMAP algorithm is described in Leland McInnes, John Healy & James Melville. UMAP: Uniform Manifold Approximation and Projection for Dimension Reduction. ARXIV, 2018; herein incorporated by reference. In some embodiments, dimensionality reduction is performed by a TriMap algorithm. The TriMap algorithm is described in Ehsan Amid & Manfred K. Warmuth TriMap: Large-scale Dimensionality Reduction Using Triplets. ARXIV, 2019; herein incorporated by reference.

In certain embodiments, the subject methods additionally include adjusting gate vertices based on an analysis of the recorded subset of classified flow cytometer data. A user may, for example, analyze the recorded subset of flow cytometer data and determine that an unacceptable number of events unrelated to the population of interest are encompassed by the first and/or second gate. In order to increase the purity of the flow cytometer data, the user may subsequently decrease the size of the gate such that the undesirable events are excluded from the boundaries of the adjusted gate. In another example, the user may analyze the recorded subset of flow cytometer data and determine that it is desirable for more events to be encompassed by the gate. In order to increase the number of events, the user may increase the size of the gate such that more events are included in the boundaries of the adjusted gate. In some cases, gate vertices are adjusted such that the shape of the gate is altered, i.e., to either include in or exclude from the gate a certain subpopulation of flow cytometer data. The vertices of the first gate, the second gate or both may be adjusted by the user, as desired. In certain instances, methods include adjusting the vertices of the first gate based on the recorded subset of classified flow cytometer data. In additional instances, methods include adjusting the vertices of the second gate based on the recorded subset of classified flow cytometer data. In still additional instances, methods include adjusting the vertices of both the first and second gates based on the recorded subset of classified flow cytometer data.

In embodiments where gate vertices are adjusted based on an analysis of the recorded subset of classified flow cytometer data, methods of interest may further include re-classifying the flow cytometer data based on the adjusted gates. Accordingly, embodiments of the subject methods include determining sets of flow cytometer data encompassed by the adjusted first gate and/or the adjusted second gate to classify the flow cytometer data. Methods may additionally include recording a subset of the classified flow cytometer data resulting from the adjusted gate(s). The recorded subset of flow cytometer data may be established by any of the mechanisms described above, such as, for example, determining a random sample of the flow cytometer data within a set difference of the set of flow cytometer data encompassed by the second gate and the flow cytometer data encompassed by the first gate.

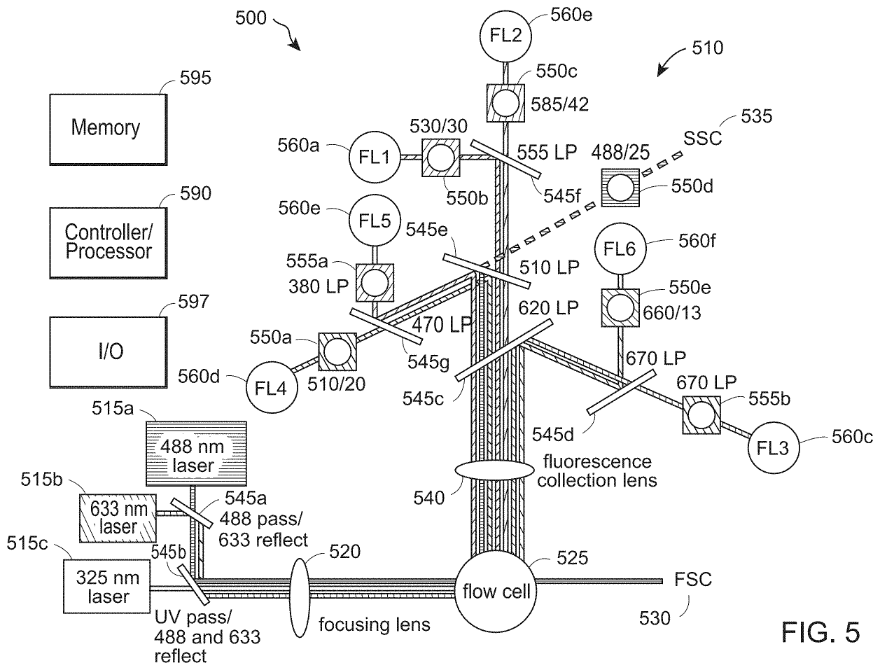

FIG. 4 provides a flowchart depicting exemplary steps for classifying flow cytometer data according to certain embodiments of the disclosure. In step 401, a user identifies a flow cytometer data population or subpopulation of interest and provides a first gate. As discussed above, the first gate may be, e.g., drawn by the user or selected from pre-established gates. In step 402, the user provides instructions that a second (i.e., expanded) gate is to be produced and provides parameters for gate expansion such as the mechanism with which the gate is to be expanded (e.g., expansion from a centroid, Minkowski addition). In step 403, the user loads a particulate sample into a flow cytometer and begins collecting flow cytometer data in real time. As flow cytometer data is generated and received in step 404, the data is classified in step 405 with respect the first and/or second gates (e.g., as described above). In step 406, the flow cytometer data encompassed by the second gate is randomly sampled, for example, by determining a random sample of the flow cytometer data within a set difference of the set of flow cytometer data encompassed by the second gate and the flow cytometer data encompassed by the first gate. In step 407, the subset of flow cytometer data determined in step 406 is subsequently recorded/saved for further analysis. After the user stops recording flow cytometer data in step 408, the user adjusts the gate vertices in step 409 based on an analysis of the recorded subset of classified flow cytometer data.

The subject methods for classifying flow cytometer data may be repeated for any desirable number of populations or subpopulations of flow cytometer data. In certain cases, flow cytometer data may possess a number of identifiable populations or subpopulations such as, for example, 2 or more populations, 3 or more populations, 4 or more populations, 5 or more populations, 6 or more populations, 7 or more populations, 8 or more populations, 9 or more populations, 10 or more populations, and including 20 or more populations. Accordingly, classification of flow cytometer data via a first gate and a second (i.e., expanded) gate may be performed with respect to any one of these populations or any combination of these populations, as desired. The classification of multiple populations of flow cytometer data may be carried out sequentially (i.e., where data in one population is classified at a time) or simultaneously (i.e., where data in multiple populations are classified at the same time. In certain cases, the classification of multiple populations of flow cytometer data is carried out sequentially. In other cases, the classification of multiple populations of flow cytometer data is carried out simultaneously.

Methods of interest may additionally include sorting particles in a sample via a sorting flow cytometer based on the first and second gates. Put another way, particles corresponding to flow cytometer data may be sorted into a series of collection vessels based on the status of the data relative to the first gate and the second gate. For example, embodiments of the method include sorting particles associated with the set of flow cytometer data encompassed by the first gate into a first collection vessel. Particles associated with flow cytometer data that are not encompassed by the first gate may, in some embodiments, be diverted to a waste collection vessel. In other embodiments, particles associated with flow cytometer data encompassed by the set difference of the set of flow cytometer data encompassed by the second gate and the set of flow cytometer data encompassed by the first gate are sorted into a second collection vessel. In certain instances, particles sorted into the second collection vessel may be considered “boundary” cases that cannot be neatly categorized but are likely to possess a sufficient number of particles of interest that it would be undesirable to discard them. Certain embodiments further include re-sorting the particles within the second collection vessel to obtain a higher yield of particles of interest. In other embodiments, all particles associated with flow cytometer data encompassed by the second gate are sorted into the same collection vessel.

Suitable collection vessels for collecting particles may include, but are not limited to: test tubes, conical tubes, multi-compartment vessels such as microtiter plates (e.g., 96-well plates), centrifuge tubes, culture tubes, microtubes, caps, cuvettes, bottles, rectilinear polymeric vessels, and bags, among other types of vessels. Particles may be sorted into any convenient number of collection vessels, such as 2 or more collection vessels, 3 or more collection vessels, 4 or more collection vessels, 5 or more collection vessels, 6 or more collection vessels, and including 7 or more collection vessels.

In some instances, the sample analyzed in the instant methods is a biological sample. The term “biological sample” is used in its conventional sense to refer to a whole organism, plant, fungi or a subset of animal tissues, cells or component parts which may in certain instances be found in blood, mucus, lymphatic fluid, synovial fluid, cerebrospinal fluid, saliva, bronchoalveolar lavage, amniotic fluid, amniotic cord blood, urine, vaginal fluid and semen. As such, a “biological sample” refers to both the native organism or a subset of its tissues as well as to a homogenate, lysate or extract prepared from the organism or a subset of its tissues, including but not limited to, for example, plasma, serum, spinal fluid, lymph fluid, sections of the skin, respiratory, gastrointestinal, cardiovascular, and genitourinary tracts, tears, saliva, milk, blood cells, tumors, organs. Biological samples may be any type of organismic tissue, including both healthy and diseased tissue (e.g., cancerous, malignant, necrotic, etc.). In certain embodiments, the biological sample is a liquid sample, such as blood or derivative thereof, e.g., plasma, tears, urine, semen, etc., where in some instances the sample is a blood sample, including whole blood, such as blood obtained from venipuncture or fingerstick (where the blood may or may not be combined with any reagents prior to assay, such as preservatives, anticoagulants, etc.).

In certain embodiments the source of the sample is a “mammal” or “mammalian”, where these terms are used broadly to describe organisms which are within the class Mammalia, including the orders carnivore (e.g., dogs and cats), Rodentia (e.g., mice, guinea pigs, and rats), and primates (e.g., humans, chimpanzees, and monkeys). In some instances, the subjects are humans. The methods may be applied to samples obtained from human subjects of both genders and at any stage of development (i.e., neonates, infant, juvenile, adolescent, adult), where in certain embodiments the human subject is a juvenile, adolescent or adult. While the present invention may be applied to samples from a human subject, it is to be understood that the methods may also be carried-out on samples from other animal subjects (that is, in “non-human subjects”) such as, but not limited to, birds, mice, rats, dogs, cats, livestock and horses.

Cells of interest may be targeted for characterized according to a variety of parameters, such as a phenotypic characteristic identified via the attachment of a particular fluorescent label to cells of interest. In some embodiments, the system is configured to deflect analyzed droplets that are determined to include a target cell. A variety of cells may be characterized using the subject methods. Target cells of interest include, but are not limited to, stem cells, T cells, dendritic cells, B Cells, granulocytes, leukemia cells, lymphoma cells, virus cells (e.g., HIV cells), NK cells, macrophages, monocytes, fibroblasts, epithelial cells, endothelial cells, and erythroid cells. Target cells of interest include cells that have a convenient cell surface marker or antigen that may be captured or labelled by a convenient affinity agent or conjugate thereof. For example, the target cell may include a cell surface antigen such as CD11 b, CD123, CD14, CD15, CD16, CD19, CD193, CD2, CD25, CD27, CD3, CD335, CD36, CD4, CD43, CD45RO, CD56, CD61, CD7, CD8, CD34, CD1c, CD23, CD304, CD235a, T cell receptor alpha/beta, T cell receptor gamma/delta, CD253, CD95, CD20, CD105, CD117, CD120b, Notch4, Lgr5 (N-Terminal), SSEA-3, TRA-1-60 Antigen, Disialoganglioside GD2 and CD71. In some embodiments, the target cell is selected from HIV containing cell, a Treg cell, an antigen-specific T-cell populations, tumor cells or hematopoietic progenitor cells (CD34+) from whole blood, bone marrow or cord blood.

Methods of interest may further include employing particles in research, laboratory testing, or therapy. In some embodiments, the subject methods include obtaining individual cells prepared from a target fluidic or tissue biological sample. For example, the subject methods include obtaining cells from fluidic or tissue samples to be used as a research or diagnostic specimen for diseases such as cancer. Likewise, the subject methods include obtaining cells from fluidic or tissue samples to be used in therapy. A cell therapy protocol is a protocol in which viable cellular material including, e.g., cells and tissues, may be prepared and introduced into a subject as a therapeutic treatment. Conditions that may be treated by the administration of the flow cytometrically sorted sample include, but are not limited to, blood disorders, immune system disorders, organ damage, etc.

A typical cell therapy protocol may include the following steps: sample collection, cell isolation, genetic modification, culture, and expansion in vitro, cell harvesting, sample volume reduction and washing, bio-preservation, storage, and introduction of cells into a subject. The protocol may begin with the collection of viable cells and tissues from source tissues of a subject to produce a sample of cells and/or tissues. The sample may be collected via any suitable procedure that includes, e.g., administering a cell mobilizing agent to a subject, drawing blood from a subject, removing bone marrow from a subject, etc. After collecting the sample, cell enrichment may occur via several methods including, e.g., centrifugation based methods, filter based methods, elutriation, magnetic separation methods, fluorescence-activated cell sorting (FACS), and the like. In some cases, the enriched cells may be genetically modified by any convenient method, e.g., nuclease mediated gene editing. The genetically modified cells can be cultured, activated, and expanded in vitro. In some cases, the cells are preserved, e.g., cryopreserved, and stored for future use where the cells are thawed and then administered to a patient, e.g., the cells may be infused in the patient.

### Systems for Classifying Flow Cytometer Data

Aspects of the invention also include systems for classifying flow cytometer data. Systems of interest include a particle analyzer (e.g., a flow cytometer) for obtaining flow cytometer data and a processor configured to classify the flow cytometer data via first and second gates. Particle analyzers of interest may include a flow cell for transporting particles in a flow stream, a light source for irradiating the particles in the flow stream at an interrogation point, and a particle-modulated light detector for detecting particle-modulated light.

As discussed herein, a “flow cell” is described in its conventional sense to refer to a component, such as a cuvette, containing a flow channel having a liquid flow stream for transporting particles in a sheath fluid. Cuvettes of interest include containers having a passage running therethrough. The flow stream may include a liquid sample injected from a sample tube. Flow cells of interest include a light-accessible flow channel. In some instances, the flow cell includes transparent material (e.g., quartz) that permits the passage of light therethrough. In some embodiments, the flow cell is a stream-in-air flow cell in which light interrogation of the particles occurs outside of the flow cell (i.e., in free space).

In some cases, the flow stream is configured for irradiation with light from a light source at an interrogation point. The flow stream for which the flow channel is configured may include a liquid sample injected from a sample tube. In certain embodiments, the flow stream may include a narrow, rapidly flowing stream of liquid that is arranged such that linearly segregated particles transported therein are separated from each other in a single-file manner. The “interrogation point” discussed herein refers to a region within the flow cell in which the particle is irradiated by light from the light source, e.g., for analysis. The size of the interrogation point may vary as desired. For example, where 0 μm represents the axis of light emitted by the light source, the interrogation point may range from −100 μm to 100 μm, such as −50 μm to 50 μm, such as −25 μm to 40 μm, and including −15 μm to 30 μm.

After particles are irradiated in the flow cell, particle-modulated light may be observed. By “particle-modulated light” it is meant light that is received from the particles in the flow stream following the irradiation of the particles with light from the light source. In some cases, the particle-modulated light is side-scattered light. As discussed herein, side-scattered light refers to light refracted and reflected from the surfaces and internal structures of the particle. In additional embodiments, the particle-modulated light includes forward-scattered light (i.e., light that travels through or around the particle in mostly a forward direction). In still other cases, the particle-modulated light includes fluorescent light (i.e., light emitted from a fluorochrome following irradiation with excitation wavelength light).

As discussed above, aspects of the invention also include a light source configured to irradiate particles passing through the flow cell at an interrogation point. Any convenient light source may be employed as the light source described herein. In some embodiments, the light source is a laser. In embodiments, the laser may be any convenient laser, such as a continuous wave laser. For example, the laser may be a diode laser, such as an ultraviolet diode laser, a visible diode laser and a near-infrared diode laser. In other embodiments, the laser may be a helium-neon (HeNe) laser. In some instances, the laser is a gas laser, such as a helium-neon laser, argon laser, krypton laser, xenon laser, nitrogen laser, CO₂ laser, CO laser, argon-fluorine (ArF) excimer laser, krypton-fluorine (KrF) excimer laser, xenon chlorine (XeCl) excimer laser or xenon-fluorine (XeF) excimer laser or a combination thereof. In other instances, the subject flow cytometers include a dye laser, such as a stilbene, coumarin or rhodamine laser. In yet other instances, lasers of interest include a metal-vapor laser, such as a helium-cadmium (HeCd) laser, helium-mercury (HeHg) laser, helium-selenium (HeSe) laser, helium-silver (HeAg) laser, strontium laser, neon-copper (NeCu) laser, copper laser or gold laser and combinations thereof. In still other instances, the subject flow cytometers include a solid-state laser, such as a ruby laser, an Nd:YAG laser, NdCrYAG laser, Er:YAG laser, Nd:YLF laser, Nd:YVO₄ laser, Nd:YCa₄O(BO₃)₃ laser, Nd:YCOB laser, titanium sapphire laser, thulim YAG laser, ytterbium YAG laser, ytterbium₂O₃ laser or cerium doped lasers and combinations thereof.

Laser light sources according to certain embodiments may also include one or more optical adjustment components. In certain embodiments, the optical adjustment component is located between the light source and the flow cell, and may include any device that is capable of changing the spatial width of irradiation or some other characteristic of irradiation from the light source, such as for example, irradiation direction, wavelength, beam width, beam intensity and focal spot. Optical adjustment protocols may include any convenient device which adjusts one or more characteristics of the light source, including but not limited to lenses, mirrors, filters, fiber optics, wavelength separators, pinholes, slits, collimating protocols and combinations thereof. In certain embodiments, flow cytometers of interest include one or more focusing lenses. The focusing lens, in one example, may be a de-magnifying lens. In still other embodiments, flow cytometers of interest include fiber optics.

Where the optical adjustment component is configured to move, the optical adjustment component may be configured to be moved continuously or in discrete intervals, such as for example in 0.01 μm or greater increments, such as 0.05 μm or greater, such as 0.1 μm or greater, such as 0.5 μm or greater such as 1 μm or greater, such as 10 μm or greater, such as 100 μm or greater, such as 500 μm or greater, such as 1 mm or greater, such as 5 mm or greater, such as 10 mm or greater and including 25 mm or greater increments.

Any displacement protocol may be employed to move the optical adjustment component structures, such as coupled to a moveable support stage or directly with a motor actuated translation stage, leadscrew translation assembly, geared translation device, such as those employing a stepper motor, servo motor, brushless electric motor, brushed DC motor, micro-step drive motor, high resolution stepper motor, among other types of motors.

The light source may be positioned any suitable distance from the flow cell, such as where the light source and the flow cell are separated by 0.005 mm or more, such as 0.01 mm or more, such as 0.05 mm or more, such as 0.1 mm or more, such as 0.5 mm or more, such as 1 mm or more, such as 5 mm or more, such as 10 mm or more, such as 25 mm or more and including at a distance of 100 mm or more. In addition, the light source may be positioned at any suitable angle relative to the flow cell, such as at an angle ranging from 10 degrees to 90 degrees, such as from 15 degrees to 85 degrees, such as from 20 degrees to 80 degrees, such as from 25 degrees to 75 degrees and including from 30 degrees to 60 degrees, for example at a 90 degree angle.

In some embodiments, light sources of interest include a plurality of lasers configured to provide laser light for discrete irradiation of the flow stream, such as 2 lasers or more, such as 3 lasers or more, such as 4 lasers or more, such as 5 lasers or more, such as 10 lasers or more, and including 15 lasers or more configured to provide laser light for discrete irradiation of the flow stream. Depending on the desired wavelengths of light for irradiating the flow stream, each laser may have a specific wavelength that varies from 200 nm to 1500 nm, such as from 250 nm to 1250 nm, such as from 300 nm to 1000 nm, such as from 350 nm to 900 nm and including from 400 nm to 800 nm. In certain embodiments, lasers of interest may include one or more of a 405 nm laser, a 488 nm laser, a 561 nm laser and a 635 nm laser.

As discussed above, particle analyzers of interest may further include one or more particle-modulated light detectors for detecting particle-modulated light intensity data. In some embodiments, the particle-modulated light detector(s) include one or more forward-scattered light detectors configured to detect forward-scattered light. For example, the subject particle analyzers may include 1 forward-scattered light detector or multiple forward-scattered light detectors, such as 2 or more, such as 3 or more, such as 4 or more, and including 5 or more. In certain embodiments, particle analyzers include 1 forward-scattered light detector. In other embodiments, particle analyzers include 2 forward-scattered light detectors.

Any convenient detector for detecting collected light may be used in the forward-scattered light detector described herein. Detectors of interest may include, but are not limited to, optical sensors or detectors, such as active-pixel sensors (APSs), avalanche photodiodes, image sensors, charge-coupled devices (CCDs), intensified charge-coupled devices (ICCDs), light emitting diodes, photon counters, bolometers, pyroelectric detectors, photoresistors, photovoltaic cells, photodiodes, photomultiplier tubes (PMTs), phototransistors, quantum dot photoconductors or photodiodes and combinations thereof, among other detectors. In certain embodiments, the collected light is measured with a charge-coupled device (CCD), semiconductor charge-coupled devices (CCD), active pixel sensors (APS), complementary metal-oxide semiconductor (CMOS) image sensors or N-type metal-oxide semiconductor (NMOS) image sensors. In certain embodiments, the detector is a photomultiplier tube, such as a photomultiplier tube having an active detecting surface area of each region that ranges from 0.01 cm² to 10 cm², such as from 0.05 cm² to 9 cm², such as from 0.1 cm² to 8 cm², such as from 0.5 cm² to 7 cm² and including from 1 cm² to 5 cm².

In embodiments, the forward-scattered light detector is configured to measure light continuously or in discrete intervals. In some instances, detectors of interest are configured to take measurements of the collected light continuously. In other instances, detectors of interest are configured to take measurements in discrete intervals, such as measuring light every 0.001 millisecond, every 0.01 millisecond, every 0.1 millisecond, every 1 millisecond, every 10 milliseconds, every 100 milliseconds and including every 1000 milliseconds, or some other interval.

In additional embodiments, the one or more particle-modulated light detector(s) may include one or more side-scattered light detectors for detecting side-scatter wavelengths of light (i.e., light refracted and reflected from the surfaces and internal structures of the particle). In some embodiments, particle analyzers include a single side-scattered light detector. In other embodiments, particle analyzers include multiple side-scattered light detectors, such as 2 or more, such as 3 or more, such as 4 or more, and including 5 or more.

Any convenient detector for detecting collected light may be used in the side-scattered light detector described herein. Detectors of interest may include, but are not limited to, optical sensors or detectors, such as active-pixel sensors (APSs), avalanche photodiodes, image sensors, charge-coupled devices (CCDs), intensified charge-coupled devices (ICCDs), light emitting diodes, photon counters, bolometers, pyroelectric detectors, photoresistors, photovoltaic cells, photodiodes, photomultiplier tubes (PMTs), phototransistors, quantum dot photoconductors or photodiodes and combinations thereof, among other detectors. In certain embodiments, the collected light is measured with a charge-coupled device (CCD), semiconductor charge-coupled devices (CCD), active pixel sensors (APS), complementary metal-oxide semiconductor (CMOS) image sensors or N-type metal-oxide semiconductor (NMOS) image sensors. In certain embodiments, the detector is a photomultiplier tube, such as a photomultiplier tube having an active detecting surface area of each region that ranges from 0.01 cm² to 10 cm², such as from 0.05 cm² to 9 cm², such as from 0.1 cm² to 8 cm², such as from 0.5 cm² to 7 cm² and including from 1 cm² to 5 cm².

In embodiments, the subject particle analyzers also include a fluorescent light detector configured to detect one or more fluorescent wavelengths of light. In other embodiments, particle analyzers include multiple fluorescent light detectors such as 2 or more, such as 3 or more, such as 4 or more, 5 or more, 10 or more, 15 or more, and including 20 or more.

Any convenient detector for detecting collected light may be used in the fluorescent light detector described herein. Detectors of interest may include, but are not limited to, optical sensors or detectors, such as active-pixel sensors (APSs), avalanche photodiodes, image sensors, charge-coupled devices (CCDs), intensified charge-coupled devices (ICCDs), light emitting diodes, photon counters, bolometers, pyroelectric detectors, photoresistors, photovoltaic cells, photodiodes, photomultiplier tubes (PMTs), phototransistors, quantum dot photoconductors or photodiodes and combinations thereof, among other detectors. In certain embodiments, the collected light is measured with a charge-coupled device (CCD), semiconductor charge-coupled devices (CCD), active pixel sensors (APS), complementary metal-oxide semiconductor (CMOS) image sensors or N-type metal-oxide semiconductor (NMOS) image sensors. In certain embodiments, the detector is a photomultiplier tube, such as a photomultiplier tube having an active detecting surface area of each region that ranges from 0.01 cm² to 10 cm², such as from 0.05 cm² to 9 cm², such as from, such as from 0.1 cm² to 8 cm², such as from 0.5 cm² to 7 cm² and including from 1 cm² to 5 cm².

Where the subject particle analyzers include multiple fluorescent light detectors, each fluorescent light detector may be the same, or the collection of fluorescent light detectors may be a combination of different types of detectors. For example, where the subject particle analyzers include two fluorescent light detectors, in some embodiments the first fluorescent light detector is a CCD-type device and the second fluorescent light detector (or imaging sensor) is a CMOS-type device. In other embodiments, both the first and second fluorescent light detectors are CCD-type devices. In yet other embodiments, both the first and second fluorescent light detectors are CMOS-type devices. In still other embodiments, the first fluorescent light detector is a CCD-type device and the second fluorescent light detector is a photomultiplier tube (PMT). In still other embodiments, the first fluorescent light detector is a CMOS-type device and the second fluorescent light detector is a photomultiplier tube. In yet other embodiments, both the first and second fluorescent light detectors are photomultiplier tubes.

In embodiments of the present disclosure, fluorescent light detectors of interest are configured to measure collected light at one or more wavelengths, such as at 2 or more wavelengths, such as at 5 or more different wavelengths, such as at 10 or more different wavelengths, such as at 25 or more different wavelengths, such as at 50 or more different wavelengths, such as at 100 or more different wavelengths, such as at 200 or more different wavelengths, such as at 300 or more different wavelengths and including measuring light emitted by a sample in the flow stream at 400 or more different wavelengths. In some embodiments, 2 or more detectors in the particle analyzers as described herein are configured to measure the same or overlapping wavelengths of collected light.

In some embodiments, fluorescent light detectors of interest are configured to measure collected light over a range of wavelengths (e.g., 200 nm-1000 nm). In certain embodiments, detectors of interest are configured to collect spectra of light over a range of wavelengths. For example, particle analyzers may include one or more detectors configured to collect spectra of light over one or more of the wavelength ranges of 200 nm-1000 nm. In yet other embodiments, detectors of interest are configured to measure light emitted by a sample in the flow stream at one or more specific wavelengths. For example, particle analyzers may include one or more detectors configured to measure light at one or more of 450 nm, 518 nm, 519 nm, 561 nm, 578 nm, 605 nm, 607 nm, 625 nm, 650 nm, 660 nm, 667 nm, 670 nm, 668 nm, 695 nm, 710 nm, 723 nm, 780 nm, 785 nm, 647 nm, 617 nm and any combinations thereof. In certain embodiments, one or more detectors may be configured to be paired with specific fluorophores, such as those used with the sample in a fluorescence assay.

In some embodiments, particle analyzers include one or more wavelength separators positioned between the flow cell and the particle-modulated light detector(s). The term “wavelength separator” is used herein in its conventional sense to refer to an optical component that is configured to separate light collected from the sample into predetermined spectral ranges. In some embodiments, particle analyzers include a single wavelength separator. In other embodiments, particle analyzers include a plurality of wavelength separators, such as 2 or more wavelength separators, such as 3 or more, such as 4 or more, such as 5 or more, such as 6 or more, such as 7 or more, such as 8 or more, such as 9 or more, such as 10 or more, such as 15 or more, such as or more, such as 50 or more, such as 75 or more and including 100 or more wavelength separators. In some embodiments, the wavelength separator is configured to separate light collected from the sample into predetermined spectral ranges by passing light having a predetermined spectral range and reflecting one or more remaining spectral ranges of light. In other embodiments, the wavelength separator is configured to separate light collected from the sample into predetermined spectral ranges by passing light having a predetermined spectral range and absorbing one or more remaining spectral ranges of light. In yet other embodiments, the wavelength separator is configured to spatially diffract light collected from the sample into predetermined spectral ranges. Each wavelength separator may be any convenient light separation protocol, such as one or more dichroic mirrors, bandpass filters, diffraction gratings, beam splitters or prisms. In some embodiments, the wavelength separator is a prism. In other embodiments, the wavelength separator is a diffraction grating. In certain embodiments, wavelength separators in the subject light detection systems are dichroic mirrors.

In some embodiments, the subject flow cytometers are operated in conjunction with programmable logic that may be implemented in hardware, software, firmware, or any combination thereof in order to classify flow cytometer data. For example, where programmable logic is implemented in software, particle classification may be realized at least in part by a computer-readable data storage medium comprising program code including instructions that, when executed, is configured to receive a first gate having a set of vertices and the flow cytometer data from the flow cytometer, expand the first gate to generate a second gate, and determine sets of flow cytometer data encompassed by each of the first gate and the second gate to classify the flow cytometer.

The programmable logic may employ any convenient mechanism may for expanding the first gate. In some embodiments, the first gate is expanded based on the distance of each gate vertex to the centroid of the gate. The centroid may be calculated by, for example, determining the arithmetic mean position of every vertex of the first gate. Following the calculation of the centroid, the programmable logic may be configured to adjust each vertex of the first gate such that the horizontal and vertical differences of the vertices from the centroid is increased by a percentage. In other words, the distance separating each vertex from the centroid is increased. In additional embodiments, expanding the first gate to generate a second gate includes Minkowski addition. In such embodiments, the set of vertices constituting the first gate is summed with the vertices of an expansion object in order to generate the second gate. In order to expand the first gate by a given magnitude, an expansion object having a particular size may be selected. In other words, the extent to which the first gate is expanded is proportional to the size of the expansion object. Any convenient geometric figure may be employed as the expansion object, including, but not limited to, a circle, ellipse, crescent, triangle, square or rectangle, as well as irregular shapes, e.g., a parabolic bottom portion coupled to a planar top portion. In some embodiments, the expansion object is an expansion circle. For example, where A represents a set of vertices (i.e., position vectors) constituting the first gate and B represents a set of vertices (i.e., position vectors) constituting the expansion object (e.g., expansion circle), the vertices of the second gate may be calculated by adding each vertex in A to each vertex in B. Put another way, the vertices of the second gate may be found in the set resulting from A+B={a+b|a∈A, b∈B}. The set of vertices may subsequently be rendered on a two-dimensional plot and connected such that the second gate is produced and visually represented.

Embodiments of the programmable logic may be further configured to record a subset of the classified flow cytometer data (e.g., as discussed in the Methods section). The recorded subset of flow cytometer data may be employed to, for example, assess the suitability of the first gate, the second gate or both for accurately gating flow cytometer data belonging to a certain population. A user may subsequently examine the recorded subset of flow cytometer data and adjust the gate boundaries accordingly.

The subject programmable logic may be implemented in any of a variety of devices such as specifically programmed event processing computers, wireless communication devices, integrated circuit devices, or the like. In some embodiments, the programable logic may be executed by a specifically programmed processor, which may include one or more processors, such as one or more digital signal processors (DSPs), configurable microprocessors, application specific integrated circuits (ASICs), field programmable gate arrays (FPGAs), or other equivalent integrated or discrete logic circuitry. A combination of computing devices, e.g., a combination of a DSP and a microprocessor, a plurality of microprocessors, one or more microprocessors in conjunction with a DSP core, or any other such configuration in at least partial data connectivity may implement one or more of the features described herein.

As discussed above, aspects of the subject flow cytometers include a flow cell configured to propagate particles in a flow stream. Any convenient flow cell which propagates a fluidic sample to a sample interrogation region may be employed, where in some embodiments, the flow cell includes is a cylindrical flow cell, a frustoconical flow cell or a flow cell that includes a proximal cylindrical portion defining a longitudinal axis and a distal frustoconical portion which terminates in a flat surface having the orifice that is transverse to the longitudinal axis.

In some embodiments, the sample flow stream emanates from an orifice at the distal end of the flow cell. Depending on the desired characteristics of the flow stream, the flow cell orifice may be any suitable shape where cross-sectional shapes of interest include, but are not limited to: rectilinear cross sectional shapes, e.g., squares, rectangles, trapezoids, triangles, hexagons, etc., curvilinear cross-sectional shapes, e.g., circles, ovals, as well as irregular shapes, e.g., a parabolic bottom portion coupled to a planar top portion. In certain embodiments, flow cell of interest has a circular orifice. The size of the nozzle orifice may vary, in some embodiments ranging from 1 μm to 10000 μm, such as from 25 μm to 7500 μm, such as from 50 μm to 5000 μm, such as from 75 μm to 1000 μm, such as from 100 μm to 750 μm and including from 150 μm to 500 μm. In certain embodiments, the nozzle orifice is 100 μm.

In some embodiments, the flow cell includes a sample injection port configured to provide a sample to the flow cell. The sample injection port may be an orifice positioned in a wall of the inner chamber or may be a conduit positioned at the proximal end of the inner chamber. Where the sample injection port is an orifice positioned in a wall of the inner chamber, the sample injection port orifice may be any suitable shape where cross-sectional shapes of interest include, but are not limited to: rectilinear cross sectional shapes, e.g., squares, rectangles, trapezoids, triangles, hexagons, etc., curvilinear cross-sectional shapes, e.g., circles, ovals, etc., as well as irregular shapes, e.g., a parabolic bottom portion coupled to a planar top portion. In certain embodiments, the sample injection port has a circular orifice. The size of the sample injection port orifice may vary depending on shape, in certain instances, having an opening ranging from 0.1 mm to 5.0 mm, such as 0.2 to 3.0 mm, such as 0.5 mm to 2.5 mm, such as from 0.75 mm to 2.25 mm, such as from 1 mm to 2 mm and including from 1.25 mm to 1.75 mm, for example 1.5 mm.

In certain instances, the sample injection port is a conduit positioned at a proximal end of the flow cell inner chamber. For example, the sample injection port may be a conduit positioned to have the orifice of the sample injection port in line with the flow cell orifice. Where the sample injection port is a conduit positioned in line with the flow cell orifice, the cross-sectional shape of the sample injection tube may be any suitable shape where cross-sectional shapes of interest include, but are not limited to: rectilinear cross sectional shapes, e.g., squares, rectangles, trapezoids, triangles, hexagons, etc., curvilinear cross-sectional shapes, e.g., circles, ovals, as well as irregular shapes, e.g., a parabolic bottom portion coupled to a planar top portion. The orifice of the conduit may vary depending on shape, in certain instances, having an opening ranging from 0.1 mm to 5.0 mm, e.g., 0.2 to 3.0 mm, e.g., 0.5 mm to 2.5 mm, such as from 0.75 mm to 2.25 mm, such as from 1 mm to 2 mm and including from 1.25 mm to 1.75 mm, for example 1.5 mm. The shape of the tip of the sample injection port may be the same or different from the cross-sectional shape of the sample injection tube. For example, the orifice of the sample injection port may include a beveled tip having a bevel angle ranging from 1 degree to 10 degrees, such as from 2 degrees to 9 degrees, such as from 3 degrees to 8 degrees, such as from 4 degrees to 7 degrees and including a bevel angle of 5 degrees.

In some embodiments, the flow cell also includes a sheath fluid injection port configured to provide a sheath fluid to the flow cell. In embodiments, the sheath fluid injection system is configured to provide a flow of sheath fluid to the flow cell inner chamber, for example in conjunction with the sample to produce a laminated flow stream of sheath fluid surrounding the sample flow stream. Depending on the desired characteristics of the flow stream, the rate of sheath fluid conveyed to the flow cell chamber by the may be 25 μL/sec to 2500 μL/sec, such as 50 μL/sec to 1000 μL/sec, and including 75 μL/sec or more to 750 μL/sec.

In some embodiments, the sheath fluid injection port is an orifice positioned in a wall of the inner chamber. The sheath fluid injection port orifice may be any suitable shape where cross-sectional shapes of interest include, but are not limited to: rectilinear cross sectional shapes, e.g., squares, rectangles, trapezoids, triangles, hexagons, etc., curvilinear cross-sectional shapes, e.g., circles, ovals, as well as irregular shapes, e.g., a parabolic bottom portion coupled to a planar top portion. The size of the sample injection port orifice may vary depending on shape, in certain instances, having an opening ranging from 0.1 mm to 5.0 mm, e.g., 0.2 to 3.0 mm, e.g., 0.5 mm to 2.5 mm, such as from 0.75 mm to 2.25 mm, such as from 1 mm to 2 mm and including from 1.25 mm to 1.75 mm, for example 1.5 mm.

In some embodiments, systems further include a pump in fluid communication with the flow cell to propagate the flow stream through the flow cell. Any convenient fluid pump protocol may be employed to control the flow of the flow stream through the flow cell. In certain instances, systems include a peristaltic pump, such as a peristaltic pump having a pulse damper. The pump in the subject systems is configured to convey fluid through the flow cell at a rate suitable for multi-photon counting of light from the sample in the flow stream. For example, the system may include a pump that is configured to flow sample through the flow cell at a rate that ranges from 1 nL/min to 500 nL/min, such as from 1 nL/min to 250 nL/min, such as from 1 nL/min to 100 nL/min, such as from 2 nL/min to 90 nL/min, such as from 3 nL/min to 80 nL/min, such as from 4 nL/min to 70 nL/min, such as from 5 nL/min to 60 nL/min and including from 10 nL/min to 50 nL/min. In certain embodiments, the flow rate of the flow stream is from 5 nL/min to 6 nL/min.

Suitable flow cytometry systems may include, but are not limited to those described in Ormerod (ed.), Flow Cytometry: A Practical Approach, Oxford Univ. Press (1997); Jaroszeski et al. (eds.), Flow Cytometry Protocols, Methods in Molecular Biology No. 91, Humana Press (1997); Practical Flow Cytometry, 3rd ed., Wiley-Liss (1995); Virgo, et al. (2012) Ann Clin Biochem. January; 49(pt 1):17-28; Linden, et. al., Semin Throm Hemost. 2004 October; 30(5):502-11; Alison, et al. J Pathol, 2010 December; 222(4):335-344; and Herbig, et al. (2007) Crit Rev Ther Drug Carrier Syst. 24(3):203-255; the disclosures of which are incorporated herein by reference. In certain instances, flow cytometry systems of interest include BD Biosciences FACSCanto™ flow cytometer, BD Biosciences FACSCanto™ II flow cytometer, BD Accuri™ flow cytometer, BD Accuri™ C6 Plus flow cytometer, BD Biosciences FACSCelesta™ flow cytometer, BD Biosciences FACSLyric™ flow cytometer, BD Biosciences FACSVerse™ flow cytometer, BD Biosciences FACSymphony™ flow cytometer, BD Biosciences LSRFortessa™ flow cytometer, BD Biosciences LSRFortessa™ X-20 flow cytometer, BD Biosciences FACSPresto™ flow cytometer, BD Biosciences FACSVia™ flow cytometer and BD Biosciences FACSCalibur™ cell sorter, a BD Biosciences FACSCount™ cell sorter, BD Biosciences FACSLyric™ cell sorter, BD Biosciences Via™ cell sorter, BD Biosciences Influx™ cell sorter, BD Biosciences Jazz™ cell sorter, BD Biosciences Aria™ cell sorter, BD Biosciences FACSAria™ II cell sorter, BD Biosciences FACSAria™ III cell sorter, BD Biosciences FACSAria™ Fusion cell sorter and BD Biosciences FACSMelody™ cell sorter, BD Biosciences FACSymphony™ S6 cell sorter or the like.

In some embodiments, the subject systems are flow cytometric systems, such those described in U.S. Pat. Nos. 10,663,476; 10,620,111; 10,613,017; 10,605,713; 10,585,031; 10,578,542; 10,578,469; 10,481,074; 10,302,545; 10,145,793; 10,113,967; 10,006,852; 9,952,076; 9,933,341; 9,726,527; 9,453,789; 9,200,334; 9,097,640; 9,095,494; 9,092,034; 8,975,595; 8,753,573; 8,233,146; 8,140,300; 7,544,326; 7,201,875; 7,129,505; 6,821,740; 6,813,017; 6,809,804; 6,372,506; 5,700,692; 5,643,796; 5,627,040; 5,620,842; 5,602,039; 4,987,086; 4,498,766; the disclosures of which are herein incorporated by reference in their entirety.

In certain instances, flow cytometry systems of the invention are configured for imaging particles in a flow stream by fluorescence imaging using radiofrequency tagged emission (FIRE), such as those described in Diebold, et al. Nature Photonics Vol. 7(10); 806-810 (2013) as well as described in U.S. Pat. Nos. 9,423,353; 9,784,661; 9,983,132; 10,006,852; 10,078,045; 10,036,699; 10,222,316; 10,288,546; 10,324,019; 10,408,758; 10,451,538; 10,620,111; and U.S. Patent Publication Nos. 2017/0133857; 2017/0328826; 2017/0350803; 2018/0275042; 2019/0376895 and 2019/0376894 the disclosures of which are herein incorporated by reference.

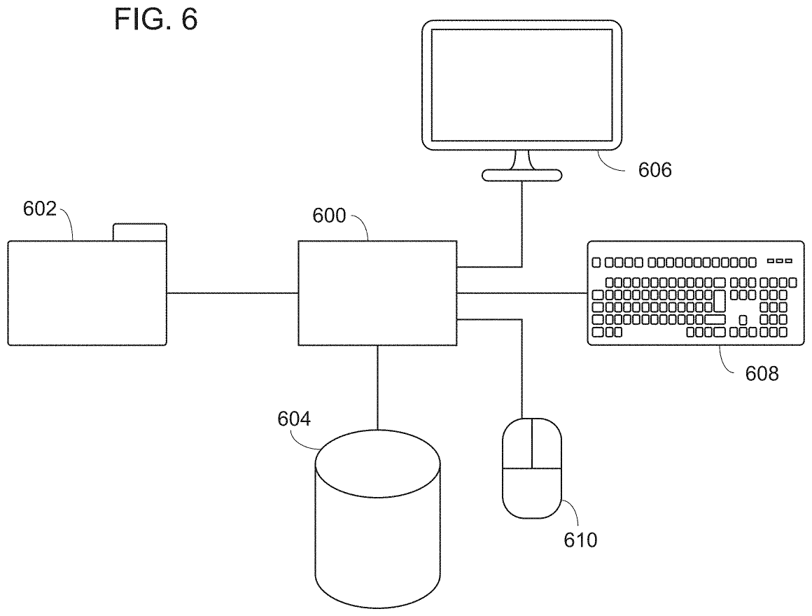

FIG. 5 shows a system 500 for flow cytometry in accordance with an illustrative embodiment of the present invention. The system 500 includes a flow cytometer 510, a controller/processor 590 and a memory 595. The flow cytometer 510 includes one or more excitation lasers 515a-515c, a focusing lens 520, a flow chamber 525, a forward-scatter detector 530, a side-scatter detector 535, a fluorescence collection lens 540, one or more beam splitters 545a-545g, one or more bandpass filters 550a-550e, one or more longpass (“LP”) filters 555a-555b, and one or more fluorescent detectors 560a-560f.

The excitation lasers 515a-c emit light in the form of a laser beam. The wavelengths of the laser beams emitted from excitation lasers 515a-515c are 488 nm, 633 nm, and 325 nm, respectively, in the example system of FIG. 5. The laser beams are first directed through one or more of beam splitters 545a and 545b. Beam splitter 545a transmits light at 488 nm and reflects light at 633 nm. Beam splitter 545b transmits UV light (light with a wavelength in the range of 10 to 400 nm) and reflects light at 488 nm and 633 nm.

The laser beams are then directed to a focusing lens 520, which focuses the beams onto the portion of a fluid stream where particles of a sample are located, within the flow chamber 525. The flow chamber is part of a fluidics system which directs particles, typically one at a time, in a stream to the focused laser beam for interrogation. The flow chamber can comprise a flow cell in a benchtop cytometer or a nozzle tip in a stream-in-air cytometer.

The light from the laser beam(s) interacts with the particles in the sample by diffraction, refraction, reflection, scattering, and absorption with re-emission at various different wavelengths depending on the characteristics of the particle such as its size, internal structure, and the presence of one or more fluorescent molecules attached to or naturally present on or in the particle. The fluorescence emissions as well as the diffracted light, refracted light, reflected light, and scattered light may be routed to one or more of the forward-scatter detector 530, the side-scatter detector 535, and the one or more fluorescent detectors 560a-560f through one or more of the beam splitters 545c-545g, the bandpass filters 550a-550e, the longpass filters 555a-555b, and the fluorescence collection lens 540.

The fluorescence collection lens 540 collects light emitted from the particle-laser beam interaction and routes that light towards one or more beam splitters and filters. Bandpass filters, such as bandpass filters 550a-550e, allow a narrow range of wavelengths to pass through the filter. For example, bandpass filter 550a is a 510/20 filter. The first number represents the center of a spectral band. The second number provides a range of the spectral band. Thus, a 510/20 filter extends 10 nm on each side of the center of the spectral band, or from 500 nm to 520 nm. Shortpass filters transmit wavelengths of light equal to or shorter than a specified wavelength. Longpass filters, such as longpass filters 555a-555b, transmit wavelengths of light equal to or longer than a specified wavelength of light. For example, longpass filter 555b, which is a 670 nm longpass filter, transmits light equal to or longer than 670 nm. Filters are often selected to optimize the specificity of a detector for a particular fluorescent dye. The filters can be configured so that the spectral band of light transmitted to the detector is close to the emission peak of a fluorescent dye.

The forward-scatter detector 530 is positioned slightly off axis from the direct beam through the flow cell and is configured to detect diffracted light, the excitation light that travels through or around the particle in mostly a forward direction. The intensity of the light detected by the forward-scatter detector is dependent on the overall size of the particle. The forward-scatter detector can include a photodiode. The side-scatter detector 535 is configured to detect refracted and reflected light from the surfaces and internal structures of the particle that tends to increase with increasing particle complexity of structure. The fluorescence emissions from fluorescent molecules associated with the particle can be detected by the one or more fluorescent detectors 560a-560f. The side-scatter detector 535 and fluorescent detectors can include photomultiplier tubes. The signals detected at the forward-scatter detector 530, the side-scatter detector 535 and the fluorescent detectors can be converted to electronic signals (voltages) by the detectors. This data can provide information about the sample.

One of skill in the art will recognize that a flow cytometer in accordance with an embodiment of the present invention is not limited to the flow cytometer depicted in FIG. 5, but can include any flow cytometer known in the art. For example, a flow cytometer may have any number of lasers, beam splitters, filters, and detectors at various wavelengths and in various different configurations.

In operation, cytometer operation is controlled by a controller/processor 590, and the measurement data from the detectors can be stored in the memory 595 and processed by the controller/processor 590. Although not shown explicitly, the controller/processor 590 is coupled to the detectors to receive the output signals therefrom, and may also be coupled to electrical and electromechanical components of the flow cytometer 510 to control the lasers, fluid flow parameters, and the like. Input/output (I/O) capabilities 597 may be provided also in the system. The memory 595, controller/processor 590, and I/O 597 may be entirely provided as an integral part of the flow cytometer 510. In such an embodiment, a display may also form part of the I/O capabilities 597 for presenting experimental data to users of the cytometer 510. Alternatively, some or all of the memory 595 and controller/processor 590 and I/O capabilities may be part of one or more external devices such as a general purpose computer. In some embodiments, some or all of the memory 595 and controller/processor 590 can be in wireless or wired communication with the cytometer 510. The controller/processor 590 in conjunction with the memory 595 and the I/O 597 can be configured to perform various functions related to the preparation and analysis of a flow cytometer experiment.

The system illustrated in FIG. 5 includes six different detectors that detect fluorescent light in six different wavelength bands (which may be referred to herein as a “filter window” for a given detector) as defined by the configuration of filters and/or splitters in the beam path from the flow cell 525 to each detector. Different fluorescent molecules used for a flow cytometer experiment will emit light in their own characteristic wavelength bands. The particular fluorescent labels used for an experiment and their associated fluorescent emission bands may be selected to generally coincide with the filter windows of the detectors. The I/O 597 can be configured to receive data regarding a flow cytometer experiment having a panel of fluorescent labels and a plurality of cell populations having a plurality of markers, each cell population having a subset of the plurality of markers. The I/O 597 can also be configured to receive biological data assigning one or more markers to one or more cell populations, marker density data, emission spectrum data, data assigning labels to one or more markers, and cytometer configuration data. Flow cytometer experiment data, such as label spectral characteristics and flow cytometer configuration data can also be stored in the memory 595. The controller/processor 590 can be configured to evaluate one or more assignments of labels to markers.

In some embodiments, the subject systems are particle sorting systems that are configured to sort particles with an enclosed particle sorting module, such as those described in U.S. Patent Publication No. 2017/0299493, filed on Mar. 28, 2017, the disclosure of which is incorporated herein by reference. In certain embodiments, particles (e.g., cells) of the sample are sorted using a sort decision module having a plurality of sort decision units, such as those described in U.S. Patent Publication No. 2020/0256781, filed on Dec. 23, 2019, the disclosure of which is incorporated herein by reference. In some embodiments, systems for sorting components of a sample include a particle sorting module having deflection plates, such as described in U.S. Patent Publication No. 2017/0299493, filed on Mar. 28, 2017, the disclosure of which is incorporated herein by reference.

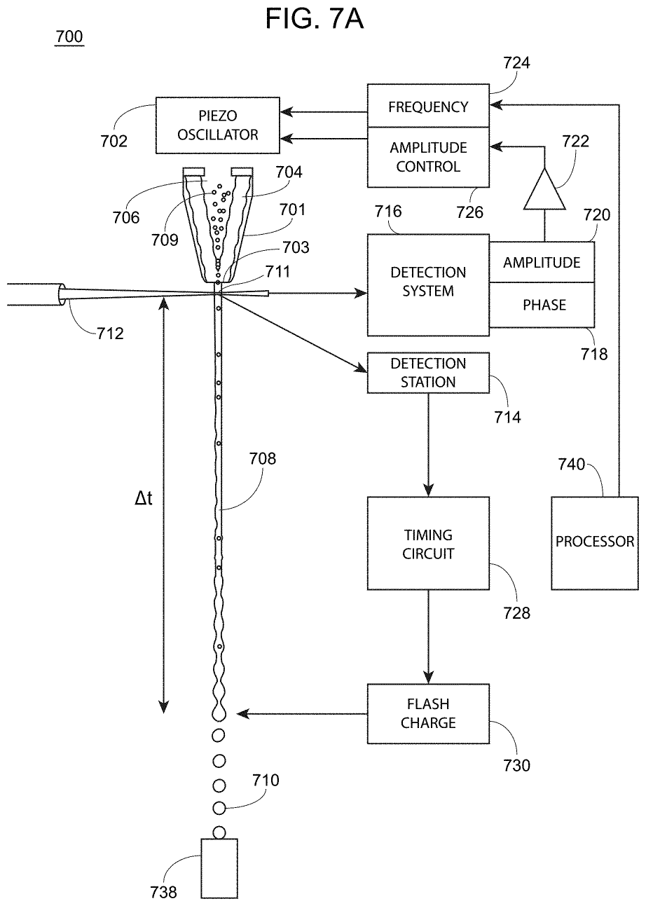

FIG. 6 shows a functional block diagram for one example of a sorting control system, such as a processor 600, for analyzing and displaying biological events. A processor 600 can be configured to implement a variety of processes for controlling graphic display of biological events.

A flow cytometer or sorting system 602 can be configured to acquire biological event data. For example, a flow cytometer can generate flow cytometric event data (e.g., particle-modulated light data). The flow cytometer 602 can be configured to provide biological event data to the processor 600. A data communication channel can be included between the flow cytometer 602 and the processor 600. The biological event data can be provided to the processor 600 via the data communication channel.

The processor 600 can be configured to receive biological event data from the flow cytometer 602. The biological event data received from the flow cytometer 602 can include flow cytometric event data. The processor 600 can be configured to provide a graphical display including a first plot of biological event data to a display device 606. The processor 600 can be further configured to render a region of interest as a gate (e.g., a first gate) around a population of biological event data shown by the display device 606, overlaid upon the first plot, for example. In some embodiments, the gate can be a logical combination of one or more graphical regions of interest drawn upon a single parameter histogram or bivariate plot. In some embodiments, the display can be used to display particle parameters or saturated detector data.

The processor 600 can be further configured to display the biological event data on the display device 606 within the gate differently from other events in the biological event data outside of the gate. For example, the processor 600 can be configured to render the color of biological event data contained within the gate to be distinct from the color of biological event data outside of the gate. The display device 606 can be implemented as a monitor, a tablet computer, a smartphone, or other electronic device configured to present graphical interfaces.

The processor 600 can be configured to receive a gate selection signal identifying the gate from a first input device. For example, the first input device can be implemented as a mouse 610. The mouse 610 can initiate a gate selection signal to the processor 600 identifying the gate to be displayed on or manipulated via the display device 606 (e.g., by clicking on or in the desired gate when the cursor is positioned there). In some implementations, the first device can be implemented as the keyboard 608 or other means for providing an input signal to the processor 600 such as a touchscreen, a stylus, an optical detector, or a voice recognition system. Some input devices can include multiple inputting functions. In such implementations, the inputting functions can each be considered an input device. For example, as shown in FIG. 6, the mouse 610 can include a right mouse button and a left mouse button, each of which can generate a triggering event.

The triggering event can cause the processor 600 to alter the manner in which the data is displayed, which portions of the data is actually displayed on the display device 606, and/or provide input to further processing such as selection of a population of interest for particle sorting.

In some embodiments, the processor 600 can be configured to detect when gate selection is initiated by the mouse 610. The processor 600 can be further configured to automatically modify plot visualization to facilitate the gating process. The modification can be based on the specific distribution of biological event data received by the processor 600. In some embodiments, the processor 600 expands the first gate such that a second gate is generated (e.g., as discussed above).

The processor 600 can be connected to a storage device 604. The storage device 604 can be configured to receive and store biological event data from the processor 600. The storage device 604 can also be configured to receive and store flow cytometric event data from the processor 600. The storage device 604 can be further configured to allow retrieval of biological event data, such as flow cytometric event data, by the processor 600.

The display device 606 can be configured to receive display data from the processor 600. The display data can comprise plots of biological event data and gates outlining sections of the plots. The display device 606 can be further configured to alter the information presented according to input received from the processor 600 in conjunction with input from the flow cytometer 602, the storage device 604, the keyboard 608, and/or the mouse 610.

In some implementations the processor 600 can generate a user interface to receive example events for sorting. For example, the user interface can include a mechanism for receiving example events or example images. The example events or images or an example gate can be provided prior to collection of event data for a sample or based on an initial set of events for a portion of the sample.

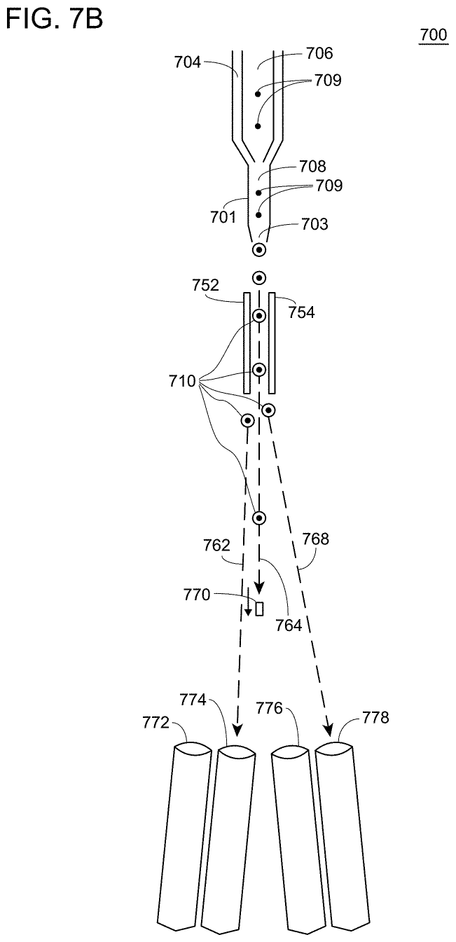

FIG. 7A is a schematic drawing of a particle sorter system 700 (e.g., the flow cytometer 602) in accordance with one embodiment presented herein. In some embodiments, the particle sorter system 700 is a cell sorter system. As shown in FIG. 7A, a drop formation transducer 702 (e.g., piezo-oscillator) is coupled to a fluid conduit 701, which can be coupled to, can include, or can be, a nozzle 703. Within the fluid conduit 701, sheath fluid 704 hydrodynamically focuses a sample fluid 706 comprising particles 709 into a moving fluid column 708 (e.g. a stream). Within the moving fluid column 708, particles 709 (e.g., cells) are lined up in single file to cross a monitored area 711 (e.g., where laser-stream intersect), irradiated by an irradiation source 712 (e.g., a laser). Vibration of the drop formation transducer 702 causes moving fluid column 708 to break into a plurality of drops 710, some of which contain particles 709.

In operation, a detection station 714 (e.g., an event detector) identifies when a particle of interest (or cell of interest) crosses the monitored area 711. Detection station 714 feeds into a timing circuit 728, which in turn feeds into a flash charge circuit 730. At a drop break off point, informed by a timed drop delay (at), a flash charge can be applied to the moving fluid column 708 such that a drop of interest carries a charge. The drop of interest can include one or more particles or cells to be sorted. The charged drop can then be sorted by activating deflection plates (not shown) to deflect the drop into a vessel such as a collection tube or a multi-well or microwell sample plate where a well or microwell can be associated with drops of particular interest. As shown in FIG. 7A, the drops can be collected in a drain receptacle 738.

A detection system 716 (e.g. a drop boundary detector) serves to automatically determine the phase of a drop drive signal when a particle of interest passes the monitored area 711. An exemplary drop boundary detector is described in U.S. Pat. No. 7,679,039, which is incorporated herein by reference in its entirety. The detection system 716 allows the instrument to accurately calculate the place of each detected particle in a drop. The detection system 716 can feed into an amplitude signal 720 and/or phase 718 signal, which in turn feeds (via amplifier 722) into an amplitude control circuit 726 and/or frequency control circuit 724. The amplitude control circuit 726 and/or frequency control circuit 724, in turn, controls the drop formation transducer 702. The amplitude control circuit 726 and/or frequency control circuit 724 can be included in a control system.

In some implementations, sort electronics (e.g., the detection system 716, the detection station 714 and a processor 740) can be coupled with a memory configured to store the detected events and a sort decision based thereon. The sort decision can be included in the event data for a particle. In some implementations, the detection system 716 and the detection station 714 can be implemented as a single detection unit or communicatively coupled such that an event measurement can be collected by one of the detection system 716 or the detection station 714 and provided to the non-collecting element.

FIG. 7B is a schematic drawing of a particle sorter system, in accordance with one embodiment presented herein. The particle sorter system 700 shown in FIG. 7B, includes deflection plates 752 and 754. A charge can be applied via a stream-charging wire in a barb. This creates a stream of droplets 710 containing particles 709 for analysis. The particles can be illuminated with one or more light sources (e.g., lasers) to generate light scatter and fluorescence information. The information for a particle is analyzed such as by sorting electronics or other detection system (not shown in FIG. 7B). The deflection plates 752 and 754 can be independently controlled to attract or repel the charged droplet to guide the droplet toward a destination collection vessel (e.g., one of 772, 774, 776, or 778). As shown in FIG. 7B, the deflection plates 752 and 754 can be controlled to direct a particle along a first path 762 toward the vessel 774 or along a second path 768 toward the vessel 778. If the particle is not of interest (e.g., does not exhibit scatter or illumination information within a specified sort range), deflection plates may allow the particle to continue along a flow path 764. Such uncharged droplets may pass into a waste receptacle such as via aspirator 770.

The sorting electronics can be included to initiate collection of measurements, receive fluorescence signals for particles, and determine how to adjust the deflection plates to cause sorting of the particles. Example implementations of the embodiment shown in FIG. 7B include the BD FACSAria™ line of flow cytometers commercially provided by Becton, Dickinson and Company (Franklin Lakes, N.J.).

### Computer-Controlled Systems

Aspects of the present disclosure further include computer-controlled systems, where the systems include one or more computers for complete automation or partial automation. In some embodiments, systems include a computer having a non-transitory computer readable storage medium with a computer program stored thereon, where the computer program when loaded on the computer includes instructions for receiving a first gate having a set of vertices and receiving flow cytometer data. Computer programs of interest further include instructions for expanding the first gate to generate a second gate and determining sets of flow cytometer data encompassed by each of the first gate and the second gate to classify the flow cytometer data.

The computer system may employ any convenient mechanism may for expanding the first gate. In some embodiments, the first gate is expanded based on the distance of each gate vertex to the centroid of the gate. The centroid may be calculated by, for example, determining the arithmetic mean position of every vertex of the first gate. Following the calculation of the centroid, embodiments further include adjusting each vertex of the first gate such that the horizontal and vertical differences of the vertices from the centroid is increased by a percentage. In other words, the distance separating each vertex from the centroid is increased. In additional embodiments, expanding the first gate to generate a second gate includes Minkowski addition. In such embodiments, the set of vertices constituting the first gate is summed with the vertices of an expansion object in order to generate the second gate. In order to expand the first gate by a given magnitude, an expansion object having a particular size may be selected. In other words, the extent to which the first gate is expanded is proportional to the size of the expansion object. Any convenient geometric figure may be employed as the expansion object, including, but not limited to, a circle, ellipse, crescent, triangle, square or rectangle, as well as irregular shapes, e.g., a parabolic bottom portion coupled to a planar top portion. In some embodiments, the expansion object is an expansion circle. For example, where A represents a set of vertices (i.e., position vectors) constituting the first gate and B represents a set of vertices (i.e., position vectors) constituting the expansion object (e.g., expansion circle), the vertices of the second gate may be calculated by adding each vertex in A to each vertex in B. Put another way, the vertices of the second gate may be found in the set resulting from A+B={a+b|a∈A, b∈B}. The set of vertices may subsequently be rendered on a two-dimensional plot and connected such that the second gate is produced and visually represented.

Embodiments of the computer system may be further configured to record a subset of the classified flow cytometer data (e.g., as discussed in the Methods section). The recorded subset of flow cytometer data may be employed to, for example, assess the suitability of the first gate, the second gate or both for accurately gating flow cytometer data belonging to a certain population. A user may subsequently examine the recorded subset of flow cytometer data and adjust the gate boundaries accordingly.

Systems may include a display and operator input device. Operator input devices may, for example, be a keyboard, mouse, or the like. The processing module includes a processor which has access to a memory having instructions stored thereon for performing the steps of the subject methods. The processing module may include an operating system, a graphical user interface (GUI) controller, a system memory, memory storage devices, and input-output controllers, cache memory, a data backup unit, and many other devices. The processor may be a commercially available processor, or it may be one of other processors that are or will become available. The processor executes the operating system and the operating system interfaces with firmware and hardware in a well-known manner, and facilitates the processor in coordinating and executing the functions of various computer programs that may be written in a variety of programming languages, such as Java, Perl, C++, Python, other high level or low level languages, as well as combinations thereof, as is known in the art. The operating system, typically in cooperation with the processor, coordinates and executes functions of the other components of the computer. The operating system also provides scheduling, input-output control, file and data management, memory management, and communication control and related services, all in accordance with known techniques. In some embodiments, the processor includes analog electronics which provide feedback control, such as for example negative feedback control.

The system memory may be any of a variety of known or future memory storage devices. Examples include any commonly available random access memory (RAM), magnetic medium such as a resident hard disk or tape, an optical medium such as a read and write compact disc, flash memory devices, or other memory storage device. The memory storage device may be any of a variety of known or future devices, including a compact disk drive, a tape drive, or a diskette drive. Such types of memory storage devices typically read from, and/or write to, a program storage medium (not shown) such as a compact disk. Any of these program storage media, or others now in use or that may later be developed, may be considered a computer program product. As will be appreciated, these program storage media typically store a computer software program and/or data. Computer software programs, also called computer control logic, typically are stored in system memory and/or the program storage device used in conjunction with the memory storage device.

In some embodiments, a computer program product is described comprising a computer usable medium having control logic (computer software program, including program code) stored therein. The control logic, when executed by the processor the computer, causes the processor to perform functions described herein. In other embodiments, some functions are implemented primarily in hardware using, for example, a hardware state machine. Implementation of the hardware state machine so as to perform the functions described herein will be apparent to those skilled in the relevant arts.

Memory may be any suitable device in which the processor can store and retrieve data, such as magnetic, optical, or solid-state storage devices (including magnetic or optical disks or tape or RAM, or any other suitable device, either fixed or portable). The processor may include a general-purpose digital microprocessor suitably programmed from a computer readable medium carrying necessary program code. Programming can be provided remotely to processor through a communication channel, or previously saved in a computer program product such as memory or some other portable or fixed computer readable storage medium using any of those devices in connection with memory. For example, a magnetic or optical disk may carry the programming, and can be read by a disk writer/reader. Systems of the invention also include programming, e.g., in the form of computer program products, algorithms for use in practicing the methods as described above. Programming according to the present invention can be recorded on computer readable media, e.g., any medium that can be read and accessed directly by a computer. Such media include, but are not limited to: magnetic storage media, such as floppy discs, hard disc storage medium, and magnetic tape; optical storage media such as CD-ROM; electrical storage media such as RAM and ROM; portable flash drive; and hybrids of these categories such as magnetic/optical storage media.

The processor may also have access to a communication channel to communicate with a user at a remote location. By remote location is meant the user is not directly in contact with the system and relays input information to an input manager from an external device, such as a computer connected to a Wide Area Network (“WAN”), telephone network, satellite network, or any other suitable communication channel, including a mobile telephone (i.e., smartphone).

In some embodiments, systems according to the present disclosure may be configured to include a communication interface. In some embodiments, the communication interface includes a receiver and/or transmitter for communicating with a network and/or another device. The communication interface can be configured for wired or wireless communication, including, but not limited to, radio frequency (RF) communication (e.g., Radio-Frequency Identification (RFID), Zigbee communication protocols, Wi-Fi, infrared, wireless Universal Serial Bus (USB), Ultra Wide Band (UWB), Bluetooth® communication protocols, and cellular communication, such as code division multiple access (CDMA) or Global System for Mobile communications (GSM).

In one embodiment, the communication interface is configured to include one or more communication ports, e.g., physical ports or interfaces such as a USB port, a USB-C port, an RS-232 port, or any other suitable electrical connection port to allow data communication between the subject systems and other external devices such as a computer terminal (for example, at a physician's office or in hospital environment) that is configured for similar complementary data communication.

In one embodiment, the communication interface is configured for infrared communication, Bluetooth® communication, or any other suitable wireless communication protocol to enable the subject systems to communicate with other devices such as computer terminals and/or networks, communication enabled mobile telephones, personal digital assistants, or any other communication devices which the user may use in conjunction.

In one embodiment, the communication interface is configured to provide a connection for data transfer utilizing Internet Protocol (IP) through a cell phone network, Short Message Service (SMS), wireless connection to a personal computer (PC) on a Local Area Network (LAN) which is connected to the internet, or Wi-Fi connection to the internet at a Wi-Fi hotspot.

In one embodiment, the subject systems are configured to wirelessly communicate with a server device via the communication interface, e.g., using a common standard such as 802.11 or Bluetooth® RF protocol, or an IrDA infrared protocol. The server device may be another portable device, such as a smart phone, Personal Digital Assistant (PDA) or notebook computer; or a larger device such as a desktop computer, appliance, etc. In some embodiments, the server device has a display, such as a liquid crystal display (LCD), as well as an input device, such as buttons, a keyboard, mouse or touch-screen.

In some embodiments, the communication interface is configured to automatically or semi-automatically communicate data stored in the subject systems, e.g., in an optional data storage unit, with a network or server device using one or more of the communication protocols and/or mechanisms described above.

Output controllers may include controllers for any of a variety of known display devices for presenting information to a user, whether a human or a machine, whether local or remote. If one of the display devices provides visual information, this information typically may be logically and/or physically organized as an array of picture elements. A graphical user interface (GUI) controller may include any of a variety of known or future software programs for providing graphical input and output interfaces between the system and a user, and for processing user inputs. The functional elements of the computer may communicate with each other via system bus. Some of these communications may be accomplished in alternative embodiments using network or other types of remote communications. The output manager may also provide information generated by the processing module to a user at a remote location, e.g., over the Internet, phone or satellite network, in accordance with known techniques. The presentation of data by the output manager may be implemented in accordance with a variety of known techniques. As some examples, data may include SQL, HTML or XML documents, email or other files, or data in other forms. The data may include Internet URL addresses so that a user may retrieve additional SQL, HTML, XML, or other documents or data from remote sources. The one or more platforms present in the subject systems may be any type of known computer platform or a type to be developed in the future, although they typically will be of a class of computer commonly referred to as servers. However, they may also be a main-frame computer, a workstation, or other computer type. They may be connected via any known or future type of cabling or other communication system including wireless systems, either networked or otherwise. They may be co-located or they may be physically separated. Various operating systems may be employed on any of the computer platforms, possibly depending on the type and/or make of computer platform chosen. Appropriate operating systems include Windows® NT®, Windows® XP, Windows® 7, Windows® 8, Windows® 10, iOS®, macOS®, Linux®, Ubuntu®, Fedora®, OS/400®, i5/OS®, IBM i®, Android™, SGI IRIX®, Oracle Solaris® and others.

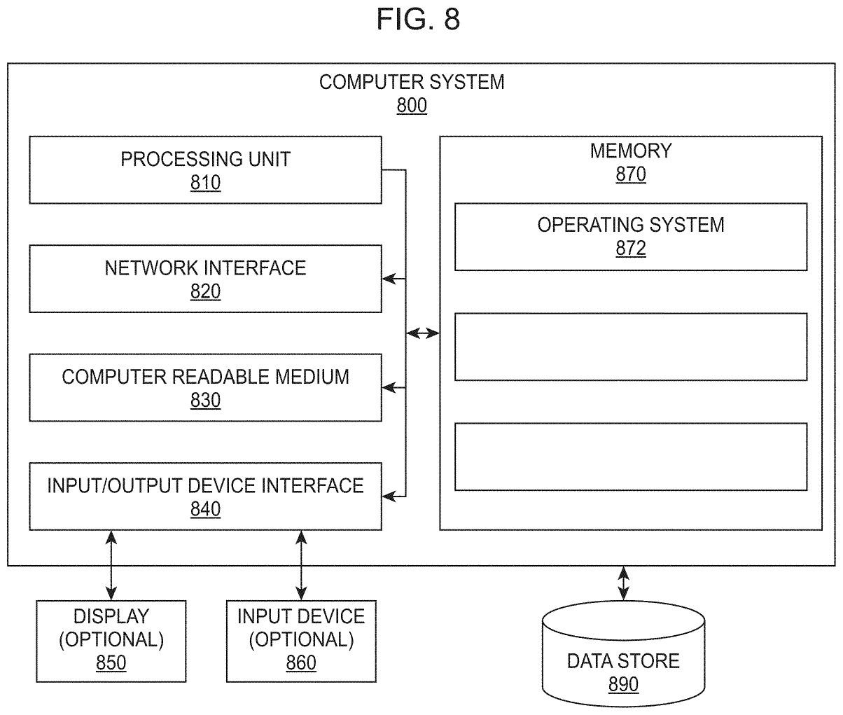

FIG. 8 depicts a general architecture of an example computing device 800 according to certain embodiments. The general architecture of the computing device 800 depicted in FIG. 8 includes an arrangement of computer hardware and software components. It is not necessary, however, that all of these generally conventional elements be shown in order to provide an enabling disclosure. As illustrated, the computing device 800 includes a processing unit 810, a network interface 820, a computer readable medium drive 830, an input/output device interface 840, a display 850, and an input device 860, all of which may communicate with one another by way of a communication bus. The network interface 820 may provide connectivity to one or more networks or computing systems. The processing unit 810 may thus receive information and instructions from other computing systems or services via a network. The processing unit 810 may also communicate to and from memory 870 and further provide output information for an optional display 850 via the input/output device interface 840. For example, an analysis software (e.g., data analysis software or program such as FlowJo®) stored as executable instructions in the non-transitory memory of the analysis system can display the flow cytometry event data to a user. The input/output device interface 840 may also accept input from the optional input device 860, such as a keyboard, mouse, digital pen, microphone, touch screen, gesture recognition system, voice recognition system, gamepad, accelerometer, gyroscope, or other input device.

The memory 870 may contain computer program instructions (grouped as modules or components in some embodiments) that the processing unit 810 executes in order to implement one or more embodiments. The memory 870 generally includes RAM, ROM and/or other persistent, auxiliary or non-transitory computer-readable media. The memory 870 may store an operating system 872 that provides computer program instructions for use by the processing unit 810 in the general administration and operation of the computing device 800. Data may be stored in data storage device 890. The memory 870 may further include computer program instructions and other information for implementing aspects of the present disclosure.

### Utility

The subject particle analyzers, methods and computer systems find use in a variety of applications where it is desirable to analyze and, optionally, sort particle components in a sample in a fluid medium, such as a biological sample, and then store sorted products, e.g., for later use, such as therapeutic use. The present invention particularly finds use where it is desirable to classify (e.g., phenotype) flow cytometer data in a certain population of flow cytometer data. For example, the subject particle analyzers, methods and computer systems may be employed to facilitate the determination of a suitable gate for a particular population or subpopulation of flow cytometer data, especially in data sets where such suitable gates are not readily apparent. Embodiments of the invention also find use where it is desirable to provide a flow cytometer with improved cell sorting accuracy, enhanced particle collection, particle charging efficiency, more accurate particle charging and enhanced particle deflection during cell sorting.

Embodiments of the invention find use in applications where cells prepared from a biological sample may be desired for research, laboratory testing or for use in therapy. In some embodiments, the subject methods and devices may facilitate obtaining individual cells prepared from a target fluidic or tissue biological sample. For example, the subject methods and systems facilitate obtaining cells from fluidic or tissue samples to be used as a research or diagnostic specimen for diseases such as cancer. Likewise, the subject methods and systems may facilitate obtaining cells from fluidic or tissue samples to be used in therapy. Methods and devices of the present disclosure allow for separating and collecting cells from a biological sample (e.g., organ, tissue, tissue fragment, fluid) with enhanced efficiency and low cost as compared to traditional flow cytometry systems.

### Kits

Aspects of the present disclosure further include kits, where kits include storage media such as a magneto-optical disk, CD-ROM, CD-R magnetic tape, non-volatile memory card, ROM, DVD-ROM, Blue-ray disk, solid state disk, and network attached storage (NAS). Any of these program storage media, or others now in use or that may later be developed, may be included in the subject kits. In embodiments, the program storage media include instructions for classifying flow cytometer data via first and second gates. In embodiments, the instructions contained on computer readable media provided in the subject kits, or a portion thereof, can be implemented as software components of a software for analyzing data. In these embodiments, computer-controlled systems according to the instant disclosure may function as a software “plugin” for an existing software package.

In addition to the above components, the subject kits may further include (in some embodiments) instructions, e.g., for installing the plugin to the existing software package. These instructions may be present in the subject kits in a variety of forms, one or more of which may be present in the kit. One form in which these instructions may be present is as printed information on a suitable medium or substrate, e.g., a piece or pieces of paper on which the information is printed, in the packaging of the kit, in a package insert, and the like. Yet another form of these instructions is a computer readable medium, e.g., diskette, compact disk (CD), portable flash drive, and the like, on which the information has been recorded. Yet another form of these instructions that may be present is a website address which may be used via the internet to access the information at a removed site.

Although the foregoing invention has been described in some detail by way of illustration and example for purposes of clarity of understanding, it is readily apparent to those of ordinary skill in the art in light of the teachings of this invention that some changes and modifications may be made thereto without departing from the spirit or scope of the appended claims.

Accordingly, the preceding merely illustrates the principles of the invention. It will be appreciated that those skilled in the art will be able to devise various arrangements which, although not explicitly described or shown herein, embody the principles of the invention and are included within its spirit and scope. Furthermore, all examples and conditional language recited herein are principally intended to aid the reader in understanding the principles of the invention and the concepts contributed by the inventors to furthering the art, and are to be construed as being without limitation to such specifically recited examples and conditions. Moreover, all statements herein reciting principles, aspects, and embodiments of the invention as well as specific examples thereof, are intended to encompass both structural and functional equivalents thereof. Additionally, it is intended that such equivalents include both currently known equivalents and equivalents developed in the future, i.e., any elements developed that perform the same function, regardless of structure. Moreover, nothing disclosed herein is intended to be dedicated to the public regardless of whether such disclosure is explicitly recited in the claims.

The scope of the present invention, therefore, is not intended to be limited to the exemplary embodiments shown and described herein. Rather, the scope and spirit of present invention is embodied by the appended claims. In the claims, 35 U.S.C. § 112(f) or 35 U.S.C. § 112(6) is expressly defined as being invoked for a limitation in the claim only when the exact phrase “means for” or the exact phrase “step for” is recited at the beginning of such limitation in the claim; if such exact phrase is not used in a limitation in the claim, then 35 U.S.C. § 112 (f) or 35 U.S.C. § 112(6) is not invoked.

1. A method of classifying flow cytometer data, the method comprising:
receiving:
a first gate comprising a set of vertices; and
flow cytometer data;
expanding the first gate to generate a second gate; and
determining sets of flow cytometer data encompassed by each of the first gate and the second gate to classify the flow cytometer data.

2. The method according to claim 1, wherein expanding the first gate to generate the second gate comprises calculating the centroid of the first gate.

3. The method according to claim 2, further comprising adjusting each vertex of the first gate such that the horizontal and vertical differences of the vertices from the centroid is increased by a percentage.

4. The method according to claim 3, wherein the percentage ranges from 1% to 20%.

5. The method according to claim 1, wherein expanding the first gate to generate the second gate comprises Minkowski addition.

6. The method according to claim 5, wherein the Minkowski addition comprises the summation of the vertices of the first gate with an expansion circle.

7. The method according to claim 1, further comprising recording a subset of the classified flow cytometer data.

8. (canceled)

9. The method according to claim 7, wherein the recorded subset of classified flow cytometer data comprises a random sample of the flow cytometer data within a set difference of the set of flow cytometer data encompassed by the second gate and the set of flow cytometer data encompassed by the first gate.

10. The method according to claim 9, wherein the random sample comprises a percentage of the classified flow cytometer data within the set difference.

11. (canceled)

12. (canceled)

13. The method according to claim 9, wherein the random sample comprises a proportion of the classified flow cytometer data within the set difference determined relative to the number of datapoints within the set of flow cytometer data encompassed by the first gate.

14. The method according to claim 7, wherein the recorded subset of classified flow cytometer data comprises a random sample of the flow cytometer data within a given distance from the first gate.

15. The method according to claim 7, wherein the recorded subset of classified flow cytometer data comprises a random sample of the universal set of flow cytometer data.

16. The method according to claim 9, wherein the recorded subset of classified flow cytometer data comprises the set union of the random sample and the set of flow cytometer data encompassed by the first gate.

17. The method according to claim 7, further comprising processing the recorded subset of classified flow cytometer with a dimensionality reduction algorithm.

18. (canceled)

19. The method according to claim 7, further comprising associating the recorded subset of classified flow cytometer data with a phenotype.

20. The method according to claim 7, further comprising adjusting the vertices of the first gate based on the recorded subset of classified flow cytometer data.

21. The method according to claim 7, further comprising adjusting the vertices of the second gate based on the recorded subset of classified flow cytometer data.

22. The method according to claim 1, further comprising differentially sorting particles in a sample via a sorting flow cytometer based on the first and second gates.

23. The method according to claim 22, further comprising sorting particles associated with the set of flow cytometer data encompassed by the first gate into a first collection vessel.

24. The method according to claim 23, further comprising sorting particles associated with flow cytometer data encompassed by the set difference of the set of flow cytometer data encompassed by the second gate and the set of flow cytometer data encompassed by the first gate into a second collection vessel.

25-94. (canceled)
